(19) United States
(12) Patent Application Publication
(10) Pub. No.: US 2024/0245511 A1
(43) Pub. Date: Jul. 25, 2024

(54) DELIVERY SYSTEM WITH ADAPTABLE NOSECONE

(71) Applicant: EDWARDS LIFESCIENCES CORPORATION, Irvine, CA (US)

(72) Inventors: Matthew Michael Becerra, Lake Forest, CA (US); David Robert Landon, Huntington Beach, CA (US); Payam Saffari, Aliso Viejo, CA (US); Yevgeniy Davidovich Kaufman, Denver, CO (US); Jesse Robert Edwards, Silverado, CA (US); David M. Taylor, Lake Forest, CA (US); Scott Louis Shary, Huntington Beach, CA (US); Richard D. White, Costa Mesa, CA (US)

(21) Appl. No.: 18/627,364

(22) Filed: Apr. 4, 2024

Related U.S. Application Data
(63) Continuation of application No. PCT/US2022/048744, filed on Nov. 2, 2022.
(60) Provisional application No. 63/275,868, filed on Nov. 4, 2021.

(51) Int. Cl. A61F 2/24

(52) U.S. Cl. CPC A61F 2/2436; A61F 2/2412

(57) ABSTRACT

A prosthetic heart valve delivery system having one or more features which facilitate access to a target annulus and improve maneuverability. The system may have a flexible access sheath having an inner lumen and a proximal handle attached to an elongated flexible catheter extending distally therefrom and having an outer diameter sized to fit through the access sheath. An expandable prosthetic heart valve is crimped and positioned in an inner lumen and near the distal end of the delivery catheter. A distal tapered nose cone attaches to the delivery catheter and facilitates passage through the patient's vasculature. An inner tube extends from the proximal handle through the delivery catheter inner lumen, through the prosthetic heart valve, and attaches to the nose cone. Finally, the system has a guidewire that extends through the proximal handle along the delivery catheter and projects distally from the nose cone. The system is especially useful for transfemoral atrioventricular valve replacements.

### CROSS REFERENCE TO RELATED APPLICATIONS

This application is a continuation of International Patent Application No. PCT/US2022/048744, filed Nov. 2, 2022, which claims the benefit of U.S. patent Application No. 63/275,868, filed Nov. 4, 2021, the entire disclosures of which are all incorporated herein by reference.

### TECHNICAL FIELD

The present application relates generally to delivery systems for implanting prostheses within a lumen or body cavity and, in particular, to delivery systems for replacement heart valves, such as replacement mitral or tricuspid heart valves.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. Delivering a prosthesis to a desired location in the human body, for example delivering a replacement heart valve to the mitral valve, can be extremely challenging. Obtaining access to perform procedures in the heart or in other anatomical locations may require delivery of devices percutaneously through tortuous vasculature. To compound the difficulty, delivery systems for prosthetic heart valves have a practical maximum diameter to enable passage through the vasculature, which limits the number and type of delivery tools that can fit within the delivery catheter.

### SUMMARY

Disclosed here is a prosthetic heart valve delivery system especially useful for transfemoral atrioventricular valve replacements. The system incorporates one or more features that facilitate access to a target annulus and improve maneuverability. The system may have a flexible access sheath having an inner lumen and a proximal handle attached to an elongated flexible delivery catheter extending distally therefrom and having an outer diameter sized to fit through the access sheath. An expandable prosthetic heart valve is crimped and positioned in an inner lumen and near the distal end of the delivery catheter. A distal tapered nose cone attaches to the delivery catheter and facilitates passage through the patient's vasculature. An inner tube extends from the proximal handle through the delivery catheter inner lumen, through the prosthetic heart valve, and attaches to the nose cone. Finally, the system has a guidewire that extends through the proximal handle along the delivery catheter and projects distally from the nose cone.

In a first aspect, a prosthetic heart valve delivery system comprises a flexible access sheath having a lumen, a proximal handle, and a delivery catheter extending distally from the proximal handle. The delivery catheter has an outer diameter sized to fit through the lumen of the access sheath, and the delivery catheter is also formed with a lumen extending therethrough. The system further includes an expandable prosthetic heart valve adapted to be crimped and positioned within the lumen of the delivery catheter along a distal end portion of the delivery catheter. A tapered nose cone couples to and projects distally from a distal end of the delivery catheter in an extended state, the nose cone being adapted to facilitate passage of the delivery catheter through the patient's vasculature. The nose cone is collapsible to a collapsed state for reducing contact with a wall of the heart, and an inner catheter extends from the proximal handle through the lumen of the delivery catheter, through the prosthetic heart valve, and attaches to the nose cone.

The nose cone may be inflatable and deflatable. In one form, the inner catheter extends a sufficient distance into the nose cone and has an inflation port open to an inflation chamber within the nose cone for inflating and deflating the nose cone. Alternatively, the nose cone is formed of braided structure that is collapsible, and the system may have a pull wire extending from the proximal handle and connected to the nose cone to cause collapse of the nose cone when pulled. Alternatively, the inner catheter attaches to a distal end of the nose cone, and the system further includes a concentric tube slidable over and relative to the inner catheter and connected to a proximal end of the nose cone, wherein relative displacement of the inner catheter and concentric tube causes collapse of the nose cone.

The inner catheter may extend through the entirety of the nose cone to a distal end thereof, and the nose cone is configured to invert upon itself when the inner catheter is pulled. In one embodiment, the nose cone is formed of an elastomeric material which may be inverted upon itself to the collapsed state. Or, the nose cone is formed of a series of stacked nested layers which form a tapered elongated shape in the extended state and which may be collapsed longitudinally in the collapsed state. The inner catheter may extend through the entirety of the nose cone to a distal end thereof, and the nose cone is configured to collapse when the inner catheter is pulled.

Another prosthetic heart valve delivery system comprises a flexible access sheath having an inner lumen, a proximal handle, and a delivery catheter having an elongated tube attached to and extending distally from the proximal handle. The elongated tube has an outer diameter sized to fit through the inner lumen of the access sheath, and also has an inner lumen extending to a distal end. An expandable prosthetic heart valve is crimped and positioned in the inner lumen of the delivery catheter near the distal end of the delivery catheter. Finally, a tapered nose cone projects distally from the distal end of the delivery catheter and is adapted to facilitate passage of the delivery catheter through the patient's vasculature. The nose cone is coupled to the distal end of the delivery catheter such that in a first configuration the nose cone projects from the distal end of the delivery catheter and in a second configuration the nose cone does not project from the distal end of the delivery catheter.

In the above system, the nose cone attaches with an interference fit to the distal end of the delivery catheter, and a retraction wire extends along the delivery catheter and connects to a distal portion of the nose cone, wherein pulling on the retraction wire displaces the nose cone laterally from the distal end of the delivery catheter to the second configuration. Alternatively, the nose cone comprises a tubular body that extends from the outside of the delivery catheter and terminates at a distal end in a retractable nose, wherein the retractable nose comprises two or more flap extensions of the tubular body which come together beyond the distal end of the delivery catheter. The tubular body is slidable over the delivery catheter such that retraction of the tubular body pulls the retractable nose in a proximal direction around the delivery catheter to the second configuration.

A third prosthetic heart valve delivery system disclosed herein comprises a flexible access sheath having an inner lumen, a proximal handle, and an elongated flexible delivery catheter having an elongated tube attached to and extending distally from the proximal handle. The elongated tube has an outer diameter sized to fit through the inner lumen of the access sheath, and also has an inner lumen extending to a distal end. An expandable prosthetic heart valve is crimped and positioned in the inner lumen of the delivery catheter near the distal end of the delivery catheter. A tapered nose cone projects distally from the distal end of the delivery catheter and is adapted to facilitate passage of the delivery catheter through the patient's vasculature. Additionally, a guidewire has a length sufficient to extend from a location proximal to the proximal handle along the delivery catheter and project distally from the nose cone. The guidewire extends along a pathway that is not centered within the delivery catheter until the distal end of the delivery catheter where the guidewire passes through an angled channel formed in the nose cone so as to project in a distal direction centrally from the nose cone. The guidewire may extend through a longitudinal passage formed in a wall of the delivery catheter prior to reaching the distal end of the delivery catheter, or the guidewire extends externally to the delivery catheter prior to reaching the distal end of the delivery catheter.

A four disclosed prosthetic heart valve delivery system comprises a flexible access sheath having an inner lumen, a proximal handle, and an elongated flexible delivery catheter having an elongated tube attached to and extending distally from the proximal handle. The elongated tube has an outer diameter sized to fit through the inner lumen of the access sheath, and also has an inner lumen extending to a distal end. An expandable prosthetic heart valve is crimped and positioned in the inner lumen of the delivery catheter near the distal end of the delivery catheter. A tapered nose cone attaches to the distal end of the delivery catheter and is adapted to facilitate passage of the delivery catheter through the patient's vasculature. Further, an inner tube extends from the proximal handle through the delivery catheter inner lumen, through the prosthetic heart valve, and attaches to the nose cone, wherein the inner tube functions as an inflation tube and is connected via the proximal handle to a source of inflation fluid.

In the fourth system embodiment, the nose cone may be is inflatable and deflatable, and the inner tube has an inflation port open to an inflation chamber within the nose cone for inflating and deflating the nose cone. Alternatively, the nose cone has a solid body surrounded by an external balloon, and the inner tube has an inflation port open to an interior of the external balloon for inflating and deflating the external balloon. Still further, the prosthetic heart valve may be balloon-expandable, and the system further includes a balloon surrounding the inner tube and within the crimped prosthetic heart valve, the inner tube having one or more side ports open to an interior space of the balloon for inflating the balloon expanding the prosthetic heart valve.

Also in the fourth system embodiment, a seal may be positioned at the distal end of the inflation tube including an elastomeric member that seals when no instruments are present. The seal may comprise a single annular member having a conical proximal lead-in wall and the central opening for passage of an instrument. Or, the seal may comprise a duck bill-type valve having two elastomeric flaps angled toward each other and projecting in a proximal direction. The seal may have a lead-in seal including an elastomeric conical member angled in a distal direction and positioned just proximal to the duck bill-type valve to facilitate passage of a guidewire through the duck bill-type valve.

The fourth system embodiment may further including a septal stabilizing balloon positioned within the delivery catheter proximal to the nose cone. The septal stabilizing balloon has a spool shape with a central circular groove sized to receive a septal wall and two annular lobes flanking the central circular groove sized to contact opposite sides of the septal wall, and the inner tube has one or more side ports open to an interior space of the septal stabilizing balloon for inflating the septal stabilizing balloon.

A still further prosthetic heart valve delivery system comprises a flexible access sheath having an inner lumen, a proximal handle, and an elongated flexible delivery catheter having an elongated tube attached to and extending distally from the proximal handle. The elongated tube has an outer diameter sized to fit through the inner lumen of the access sheath, and also has an inner lumen extending to a distal end. An expandable prosthetic heart valve is crimped and positioned in the inner lumen of the delivery catheter near the distal end of the delivery catheter. A tapered nose cone projects distally from the distal end of the delivery catheter and is adapted to facilitate passage of the delivery catheter through the patient's vasculature. Also, a guidewire having a length sufficient to extend from a location proximal to the proximal handle along the delivery catheter and project distally from the nose cone comprises a central core surrounded by an insulated outer coil. The central core and outer coil are electrically connected at a distal end of the guidewire to form a circuit and are connected to opposite poles of the source of electricity to selectively initiate a current through the circuit. A discrete section of the central core of the guidewire may be configured to convert from a flexible to a stiffer configuration upon initiation of current and consequent heating of the guidewire. The guidewire may terminate in a distal atraumatic pigtail, with the discrete section located just proximal to the pigtail.

A sixth prosthetic heart valve delivery system disclosed herein comprises a flexible access sheath having an inner lumen, the access sheath having a wall construction that enables conversion between a stiff configuration to a flexible configuration. An elongated flexible delivery catheter has an elongated tube attached to and extending distally from a proximal handle, the elongated tube having an outer diameter sized to fit through the inner lumen of the access sheath, the elongated tube also having an inner lumen extending to a distal end. An expandable prosthetic heart valve is crimped and positioned in the inner lumen of the delivery catheter near the distal end of the delivery catheter. Finally, a tapered nose cone attaches to the distal end of the delivery catheter and is adapted to facilitate passage of the delivery catheter through the patient's vasculature. The access sheath may comprise an inner tubular member surrounded by an inflatable filament coiled around the tubular member, wherein the filament is connected to a source of fluid to convert the filament from a deflated state to an inflated state and therefore stiffen the access sheath to the stiff configuration. The inner tubular member may have longitudinal pleats which enable radial compression of the access sheath when the filament is in its deflated state.

A seventh prosthetic heart valve delivery system features a flexible access sheath having an inner lumen, the access sheath having a wall construction that enables conversion between an extended configuration to an axially collapsed configuration. An elongated flexible delivery catheter having an elongated tube attaches to and extends distally from a proximal handle, the elongated tube having an outer diameter sized to fit through the inner lumen of the access sheath, the elongated tube also having an inner lumen extending to a distal end. An expandable prosthetic heart valve is crimped and positioned in the inner lumen of the delivery catheter near the distal end of the delivery catheter, and a tapered nose cone attaches to the distal end of the delivery catheter and is adapted to facilitate passage of the delivery catheter through the patient's vasculature.

In the seventh system, the access sheath may comprise an axially compressible structure within an outer jacket comprising a series of axially spaced rings joined with a plurality of axially-compressible struts between adjacent rings. For instance, the axially-compressible struts may have a serpentine or zig-zag configuration. The axially-compressible struts between any two pairs of adjacent rings may be rotationally offset between the pairs of rings in series along the access sheath.

A still further eight prosthetic heart valve delivery system comprises an elongated flexible delivery catheter having an elongated tube attached to and extending distally from a proximal handle, the elongated tube having an outer diameter and an inner lumen extending to a distal end. An expandable prosthetic heart valve is crimped and positioned within the inner lumen of the delivery catheter near the distal end of the delivery catheter. Furthermore, a break-away tip fitted over the delivery catheter comprising a flexible tubular bag having seals on distal and proximal ends in contact with an exterior of the delivery catheter, and a tapered distal end with petals. The break-away tip forms a hemostatic barrier around the delivery catheter, wherein the petals are adapted to flex outward upon distal advancement of the delivery catheter relative to the break-away tip to permit the delivery catheter to be advanced from within the break-away tip. The break-away tip may have an outward flange on a proximal end configured to contact an exterior of a patient access site and halt further distal movement of the break-away tip. The seals on distal and proximal ends are desirably O-rings.

A ninth prosthetic heart valve delivery system disclosed herein comprises an elongated flexible delivery catheter having an elongated tube attached to and extending distally from a proximal handle, the elongated tube having an outer diameter and an inner lumen extending to a distal end. An expandable prosthetic heart valve is crimped and positioned within the inner lumen of the delivery catheter near the distal end of the delivery catheter. A flexible access sheath has an inner lumen, wherein the delivery catheter is sized to fit through the inner lumen of the access sheath. Finally, a break-away tip fitted over the access sheath and delivery catheter comprises a flexible tubular bag having a proximal seal on a proximal end in contact with an exterior of the access sheath and a distal seal on a distal end in contact with an exterior of the delivery catheter, and a tapered distal end with petals. The break-away tip forms a hemostatic barrier around the access sheath and delivery catheter, wherein the petals are adapted to flex outward upon distal advancement of the delivery catheter relative to the break-away tip to permit the delivery catheter to be advanced from within the break-away tip.

In any of the preceding systems, the delivery catheter may include a motor within the proximal handle and pull wires extending from the proximal handle to a distal tip of the elongated tube and connected to steer the catheter by deflecting the distal tip in multiple directions. The motorized system may further include a control device configured to control operation of the motor including an input device, an output device, a memory and a processor, the control device being connected to a power source.

In any of the preceding systems, a catheter positioning sensor may be inserted alongside the delivery catheter to the tricuspid annulus, the sensor having a node on a distal end configured to emit an RF field. Wherein the delivery catheter has a sensor positioned to be recognized by the emitter such that a relative position of the delivery catheter sensor can be transferred to a user display. The node may be a single node point emitter an adjustable ring emitter, or an adjustable ring emitter.

In any of the preceding systems, the prosthetic heart valve may include at least one access port extending axial therethrough for passage of a wire lead without passing through valve leaflets.

A further understanding of the nature and advantages of the invention will become apparent by reference to the remaining portions of the specification and drawings.

### BRIEF DESCRIPTION OF THE DRAWINGS

Features and advantages of the present invention will become appreciated as the same become better understood with reference to the specification, claims, and appended drawings wherein:

FIG. 1 is a schematic representation of a transvascular method of introducing a flexible catheter into the heart to perform procedures, such as a valve replacement procedure at the mitral annulus, and FIG. 1A is an enlargement of just the heart showing a similar catheter having been advanced through the vasculature to the tricuspid annulus;

FIGS. 2A-2D are cross-sections of a heart showing a sequence of steps in a typical mitral valve replacement procedure utilizing a valve delivery system;

FIG. 3 is a longitudinal sectional view through the distal section of a conventional valve delivery system;

FIGS. 4A and 4B are sectional views of an inflatable nose cone that may be used with the valve delivery system of FIG. 3, in inflated and deflated configurations, respectively;

FIG. 5A illustrates an alternative collapsible nose cone formed of braided material, and FIGS. 5B and 5C show different ways to collapse the nose cone;

FIGS. 6A and 6B illustrate a collapsible/invertible nose cone in, respectively, expanded and inverted configurations;

FIG. 7A shows a removable nose cone secured to a distal end of a delivery catheter, and FIG. 7B shows one way to remove the nose cone;

FIGS. 8A and 8B illustrate a retractable nose cone;

FIG. 9 is a radial sectional view taken along line 9-9 of FIG. 3 through a crimped valve held within a valve delivery system, and showing conventional placement of a guidewire therethrough;

FIGS. 10A and 10B are radial sectional views similar to that of FIG. 9 showing alternative pathways for a guidewire through the valve delivery systems;

FIG. 11 is a longitudinal sectional view through an alternative nose cone adapted to provide an angled pathway for a guidewire routed outside a delivery catheter, such as seen in FIG. 10B;

FIGS. 12A and 12B are schematic views of the distal end of a delivery catheter showing a further alternative nose cone in extended and collapsed states;

FIGS. 13A and 13B show the distal end of the delivery catheter and a still further collapsible nose cone in both extended and collapsed states;

FIG. 14 is a longitudinal sectional view through an inflatable nose cone illustrating a distal seal within a guidewire tube which doubles as an inflation lumen;

FIGS. 15A and 15B are enlargements of a distal end of the inflatable nose cone illustrating alternative versions of the distal seal;

FIG. 16A is an enlargement of the distal end of an alternative nose cone showing an alternative distal seal, FIG. 16B shows passage of a guidewire through the seal, and FIG. 16C shows the addition of a lead-in seal which facilitates passage of the guidewire;

FIG. 17 is a schematic illustration of a system for providing fluid to a guidewire tube that doubles as an inflation tube;

FIGS. 18A and 18B are longitudinal sectional views of a still further nose cone having an outer balloon that may be inflated and deflated via the dual guidewire/inflation tube;

FIG. 19 is a longitudinal sectional view through the distal section of a valve delivery system where a portion of the crimped valve has been removed to illustrate a dual guidewire/inflation tube that may be used to inflate a valve expansion balloon;

FIG. 20 illustrates expansion of a balloon within an expandable prosthetic heart valve, such as one similar to that shown being implanted in the sequence of FIGS. 2A-2C;

FIG. 21 is a longitudinal sectional view through the distal section of the valve delivery system in which a septal stabilizing balloon is incorporated;

FIGS. 22A-22C are cross-sections of a heart showing a sequence of steps in a deploying the septal stabilizing balloon of FIG. 21;

FIG. 23 illustrates an exemplary convertible guidewire having a portion that may be stiffened when desired;

FIGS. 24A-24C illustrate a sequence of usage of the convertible guidewire of FIG. 23;

FIG. 25 is a schematic representation of a transvascular method of introducing a flexible catheter into the heart to perform procedures, showing a tortuous vasculature pathway which presents challenges to the procedure;

FIG. 26 is a view of an entire valve delivery system illustrating relative movement between a control handle and inner catheter with respect to an access sheath;

FIG. 27 illustrates a convertible access sheath such as would be used in the system of FIG. 26 having an inflatable stiffening spiral thereon;

FIGS. 28A-28C are radial sectional views showing alternative configurations of the convertible access sheath of FIG. 27;

FIG. 29 is a schematic view of a proximal end of the convertible access sheath illustrating the capacity for axial compression;

FIG. 30 is an elevational view of an alternative compressible access sheath in an extended configuration, and FIG. 30A is an enlargement of a portion thereof;

FIG. 31 is an elevational view of the compressible access sheath of FIG. 30 showing axial compression thereof;

FIGS. 32A and 32B are enlargements of several links in an alternative compressible access sheath in extended and axially compressed configurations, respectively;

FIG. 33 is a view of an entire valve delivery system illustrating forward movement of a control handle and inner catheter along with an access sheath;

FIG. 34 illustrates a distal end of the access sheath with a break-away delivery system tip incorporated thereon, and FIG. 34A shows an alternative where the break-away delivery system tip forms the access sheath;

FIG. 35 shows the break-away delivery system tip, and FIGS. 36A-36B show distal displacement of the access sheath through the tip;

FIG. 37 illustrates a steerable delivery catheter capable of multiple planes of deflection and having motorized and sensor-influenced control, and FIG. 37A is an enlargement of a distal tip thereof;

FIG. 38 illustrates a cross section of a control handle of the steerable delivery catheter including a motor, and FIG. 39 is a cross-section of an elongate shaft of the catheter showing axially extending pull wires therein;

FIGS. 40 and 41 illustrate use of a catheter positioning sensor deployed adjacent to a target tricuspid annulus;

FIG. 42 illustrates a modified prosthetic heart valve implanted at a tricuspid annulus and having through holes for passage of a sensor wire; and

FIGS. 43A and 43B are perspective and plan views of the modified prosthetic heart valve.

### DETAILED DESCRIPTION OF CERTAIN EMBODIMENTS

The right ventricle and left ventricle are separated from the right atrium and left atrium, respectively, by the tricuspid valve and mitral valve; i.e., the atrioventricular valves. The septal wall extends between the right atrium and left atrium. The present specification and drawings provide aspects and features of the disclosure in the context of several embodiments of replacement heart valves, delivery systems and methods that are configured for use in the vasculature of a patient, such as for replacement of natural heart valves in a patient. Valve replacement in the mitral or tricuspid annulus is a primary focus of the present application, but certain characteristics of the delivery systems described herein may equally be used for other valve implant locations, and thus the claims should not be constrained to mitral or tricuspid valve replacement unless expressly limited.

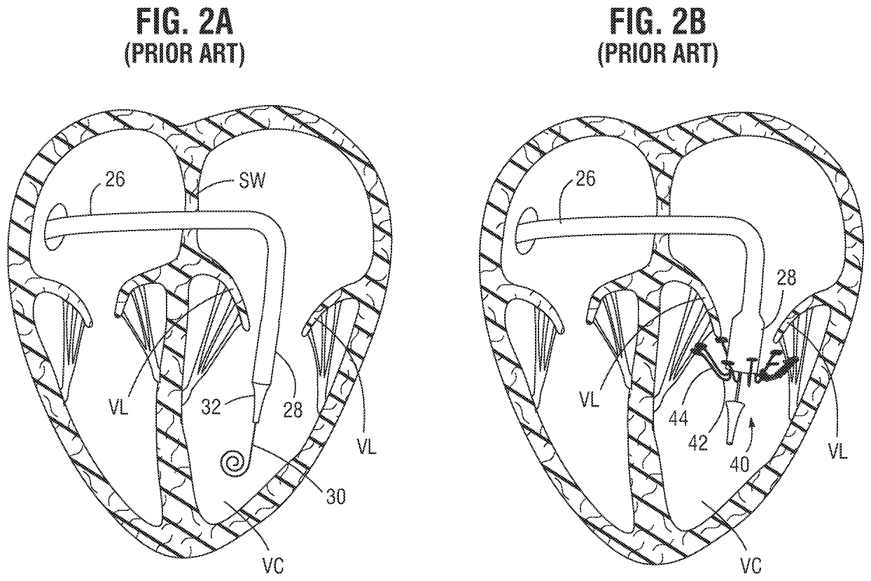

In particular, prosthetic valve delivery systems are described herein for transfemoral percutaneous delivery of a replacement mitral valve to treat patients with moderate to severe mitral regurgitation. In some cases, for safety and/or other reasons, the disclosed prosthetic devices may be delivered from the atrial side of the atrioventricular valve annulus. For example, a transatrial approach can be made through an atrial wall, which can be accessed, for example, by an incision through the chest. Atrial delivery can also be made intravascularly, such as from a pulmonary vein. The prosthetic valve can be delivered to the right atrium via the inferior or superior vena cava. In some cases, left atrial delivery can be made via a transeptal approach (FIGS. 2A-2D). In a transeptal approach, an incision can be made in the atrial portion of the septal wall SW to allow access to the left atrium from the right atrium. The prosthetic valve can also be delivered via transventricular, transatrial, or transfemoral approaches with small or minimal modifications to the delivery process.

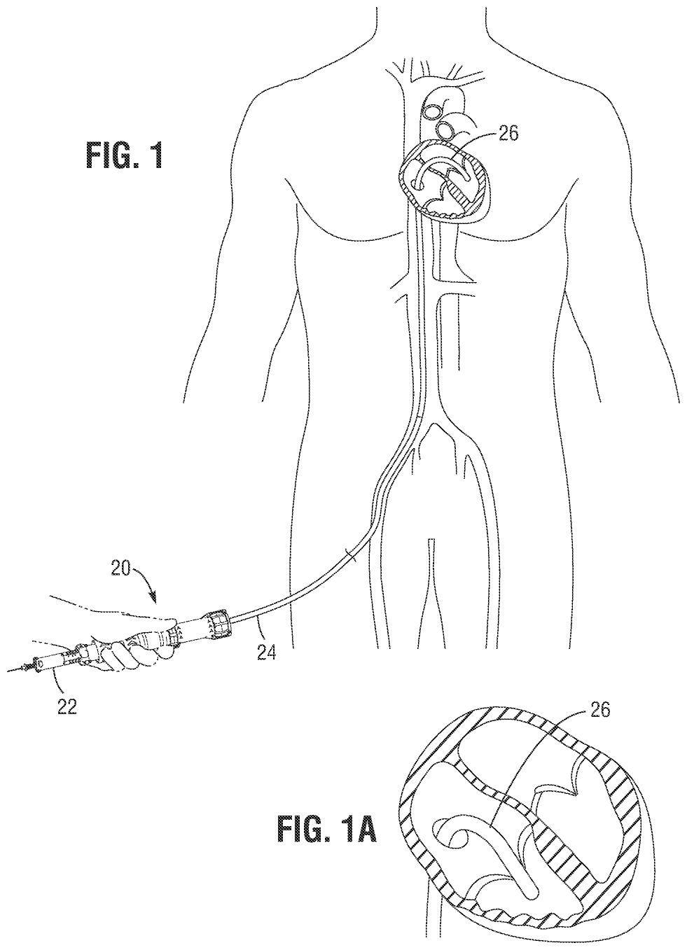

FIG. 1 illustrates an embodiment of a delivery device, assembly, or system 20. The delivery system 20 can be used to deploy a prosthesis, such as a replacement heart valve, within the body. Replacement heart valves can be delivered to a patient's heart mitral valve annulus or other heart valve location in various manners, such as by open surgery, minimally-invasive surgery, and percutaneous or transcatheter delivery through the patient's vasculature. Example transfemoral approaches may be found in U.S. Pat. Nos. 10,004,599 and 10,813,757, the entireties of which are hereby incorporated by reference. While the delivery system 20 is described in connection with a percutaneous delivery approach, and more specifically a transfemoral delivery approach, it should be understood that features of delivery system 20 can be applied to other delivery system, including delivery systems for a transapical delivery approach.

The valve delivery system 20 has a proximal handle 22 from which an elongated access sheath 24 extends distally. The access sheath 24 is shown extending into a lower portion of the venous system, such as into an ipsilateral femoral vein, and a delivery catheter 26 advances from within the access sheath 24 up through the patient's venous system into the right atrium to access the tricuspid valve, or with a further transseptal puncture using known techniques into the left atrium to access the mitral valve. FIG. 1 shows the latter procedure where a distal tip 28 (see FIG. 2A) of the delivery catheter 26 has crossed the septal wall SW, while FIG. 1A shows the distal tip in the right atrium. Both procedures are enhanced by the various delivery system attributes described herein, individually or in conjunction with one another. All combinations of structural features as described herein are contemplated, assuming they are not mutually exclusive or redundant in deployment.

It should be noted that the access sheath 24 may be integrally associated with the proximal handle 22 or may be a separate instrument. In this context, an integral sheath 24 would be fixedly attached to the proximal handle 22, with the delivery catheter 26 extending through and movable with respect to both the handle 22 and the sheath. With a separate sheath 24, the sheath would have a proximal hub with elastomeric valves, and the delivery catheter 26 is fixedly attached to the proximal handle 22 and passed through the valves to prevent blood leakage. Both types of access sheath 24 are contemplated herein, and the claims should not be considered limited to one or the other unless specifically recited.

It should be noted that the proximal handle 22 as well as attendant support systems such as guidewires and inflation connections are generally known in the art. Indeed, the proximal handle 22 may be closely structurally similar to that used in the transfemoral EVOQUE Tricuspid Valve Replacement System and the EVOQUE Transcatheter Mitral Valve Replacement System, both being developed by Edwards Lifesciences of Irvine, CA. Aspects of the proximal handle 22 are seen in U.S. Pat. Nos. 10,004,599 and 10,813,757, mentioned above.

### Exemplary Transvascular Heart Valve Delivery

To better understand certain aspects of the improvements disclosed herein, a typical mitral valve replacement procedure utilizing the valve delivery system 20 will be described with reference to FIGS. 2A-2D. The figures are labeled “PRIOR ART,” as this procedure is known in the art, though the same procedure can be practiced with one or more of the advantageous features described herein, as will be mentioned in context below.

To deliver the prosthetic valve to the native mitral valve annulus, the prosthetic valve can be radially crimped into a collapsed configuration within a delivery catheter 26 of the delivery system 20. In some embodiments, the prosthetic valve can fit inside of a 30 French (F) catheter (in a collapsed state). In some embodiments, the prosthetic valve can be configured to fit into even smaller catheters, such as a 29 F, 28 F, 27 F, or 26 F catheter.

With reference to both FIGS. 1 and 2A, the access sheath 24 of the delivery system 20 can be placed in the ipsilateral femoral vein and the delivery catheter 26 advanced through the sheath toward the right atrium. A transseptal puncture using known techniques can then be performed to obtain access to the left atrium. The delivery catheter 26 can then be advanced into the left atrium and then to the left ventricle. A guidewire 30 may be necessary to position the delivery catheter 26 in the proper position, and one or more guidewires may be used. In addition, a generally conical or otherwise tapered nose cone 32 is traditionally secured at the distal end 28 of the delivery catheter 26 to help transit through the vasculature and other obstacles, such as the septal wall SW, as well as through the valve leaflets VL into the associated ventricular cavity VC.

It can be advantageous for a user to be able to steer the delivery system 20 through the complex areas of the heart in order to position a replacement mitral valve in line with the native mitral valve. For example, a user can manipulate the distal end 28 of the delivery catheter 26 to the appropriate area by steering or bending. A user can then continue to pass the bent delivery system 20 through the transseptal puncture and into the left atrium and can then further manipulate the delivery system 20 to create an even greater bend in the delivery catheter 26. Further, a user can torque the entire delivery system 20 to further manipulate and control the position of the distal end 28. The delivery catheter 26 is further advanced such that the delivery catheter 26 (carrying the prosthetic valve) extends between the native leaflets of the mitral valve and into the left ventricle VC.

FIGS. 2B-2C show an exemplary prosthetic valve delivery, including the process of expanding a prosthetic valve 40 using the delivery catheter 26, with reference to an embodiment of a prosthetic valve having a self-expanding frame, although the delivery assembly and method are applicable to other frame embodiments such as balloon-expandable frames. In the delivery configuration (FIG. 2A), the delivery catheter 26 was previously advanced over the collapsed prosthetic valve to convert the valve to a radially collapsed configuration. Although not shown, the prosthetic heart valve 40 is typically mounted on the distal end of a delivery catheter having the capacity to displace the valve with respect to the delivery catheter 26, or vice versa. As mentioned, such a delivery catheter may incorporate an expansion balloon if the valve is balloon-expandable.

FIG. 2B shows an initial phase of expanding the prosthetic valve by retracting the delivery catheter 26 from the nose cone 32, enabling eventual expulsion of the valve. As mentioned, the nose cone 32 is useful in facilitating access of the delivery catheter 26 through the entire delivery system (through hemostatic valves and the like), as well as through the vasculature, potentially though the septal wall SW, and through whichever valve annulus in which the prosthetic valve is being implanted. The nose cone 32 is somewhat sharp and elongated which reduces the amount of space available in the ventricle for manipulation of the distal end of the catheter and placement of the valve. Indeed, some patients have such a small space within the affected ventricle, especially the right ventricle, that they are screened out of having this particular procedure. Therefore, reduction of the size of the nose cone 32 after it is useful is one of the aims of the present application.

FIG. 2B indicates partial retraction of the delivery catheter 26 to expulse a plurality of ventricular anchors 42 in a circumferential array from a distal end of the prosthetic valve. The heart valve 40 is displaced relative to the delivery catheter 26 such as by advancing a pusher device distally against the prosthetic valve and/or retracting the delivery catheter relative to the valve. Terminal end portions 44 of the anchors 42 are biased to extend proximally (in the direction of a main body of the valve) when deployed. It should be understood that the constraining or restraining force applied by the delivery catheter 26 on the anchors 42 can force the terminal end portions 44 to extend downward (away from the main body in a generally distal direction) during delivery.

Once the prosthetic valve 40 is delivered to the native annulus region, the delivery catheter 26 can be retracted farther relative to the prosthetic valve 40, thereby allowing the prosthetic valve 40 to expand radially outward. The release of the prosthetic valve 40 can be conducted in stages. In particular, the ventricular anchors 42 can be released from the delivery catheter 26 (FIG. 2B) prior to the release of a main body portion of the valve 40, as seen in FIG. 2C. When the ventricular anchors 42 are released, they spread out away from the main body, with distal end portions 44 directed radially outward and upward. Subsequently, with release of the main body, the anchors 42 rotate toward the main body, such that the distal end portions 44 pivot toward the vertical (longitudinal) axis and wrap around behind the native leaflets VL on the ventricular side.

The surgeon then optionally repositions the partially retracted valve 40 as desired, and retracts the delivery catheter 26 further to cause the ventricular anchors 42 to engage the native valve annulus (FIG. 2C). Rounded head portions 46 of the ventricular anchors 42 can contact a ventricular side of the native valve annulus and/or adjacent tissue (such as trigone areas). In particular, the anchors 42 can be configured to point more directly upward upon full deployment, as compared to when they are partially deployed from the delivery catheter 26. At this point, the user can assess engagement of the ventricular anchors 42 with the native valve annulus (such as through imaging means), prior to retracting the delivery catheter 26 farther to deploy an atrial portion 48, as in FIG. 2D.

In some implementations, one or more ventricular anchors 42 engage the chordae tendineae, one or more ventricular anchors engage the trigone areas, and/or one or more ventricular anchors engage the native leaflets at A2 and/or P2 positions (i.e., between the commissure of the native leaflets). The ventricular anchors that engage the native leaflets and the trigone areas can capture or “sandwich” the native tissue between the outer surface of the main body of the prosthetic valve and the ventricular anchors (or portions thereof) such that the tissue is compressed and engaged by the main body of the prosthetic valve on one side and by the ventricular anchors on the other side. In some embodiments, due to the capturing of the native tissue (such as the native leaflets) between the ventricular anchors and the main body, the native tissue forms a seal around the main body (through 360 degrees) within the left ventricle that impedes blood from traveling along the outside of the main body. By virtue of their relatively thin profile and because the ventricular anchors are not interconnected to each other, the distal ends of the ventricular anchors adjacent the chordae tendineae can pass between individual chords extending from the native leaflets, allowing those anchors to flex/pivot upwardly and assume their fully deployed positions.

Finally, as shown in FIG. 2D, the delivery catheter 26 is retracted farther to release the atrial portion 48 of the prosthetic valve 40. The atrial portion 48 forms a seal against the native annulus within the left atrium. The seal created in the left atrium by the atrial portion 48 and the seal created by the ventricular anchors 42 in the left ventricle together prevent, reduce, or minimize the flow of blood between the native annulus and the outside of the main body during diastole and systole. In some embodiments, the main body and the atrial portion 48 are released simultaneously, while in other embodiments, the main body is released prior to the atrial portion 48. Upon full deployment of the ventricular anchors 42 and the main body, the distal end portions 44 can be positioned against the native valve annulus and/or adjacent tissue (e.g., trigone areas). All stages of deployment of the prosthetic valve 40 can thus be controlled by the delivery catheter 26 without additional activation or manipulation necessary.

### Challenges with Transvascular Heart Valve Delivery

The foregoing discussion of a transvascular method of delivery of a heart valve to a mitral annulus is provided for context in terms of various challenges that have been discovered during clinical investigations and commercial performance of the various systems available. In general, there is an inherent friction between negotiating the sometimes complex and tortuous geometry of the vascular system and further into the heart structures against providing relatively large expandable prosthetic heart valves and attendant delivery instruments. One problem is that containment region within the delivery catheter system is extremely limited. Another problem stems from the relatively small spaces available within the heart for manipulation of the valve expansion and related features. These generalized problems become real-world issues that limit the effectiveness and ease of use of valve delivery system.

### Nose Cone Alternatives

One problem that has been identified is that the leading nose cone, seen at 32 in FIG. 2A, is typically pointed and elongated, which creates the potential for damaging internal heart structures as well as reducing the amount of space available for manipulation of the distal end of the catheter and placement of the valve. Various alternative nose cones are thus contemplated, as explained.

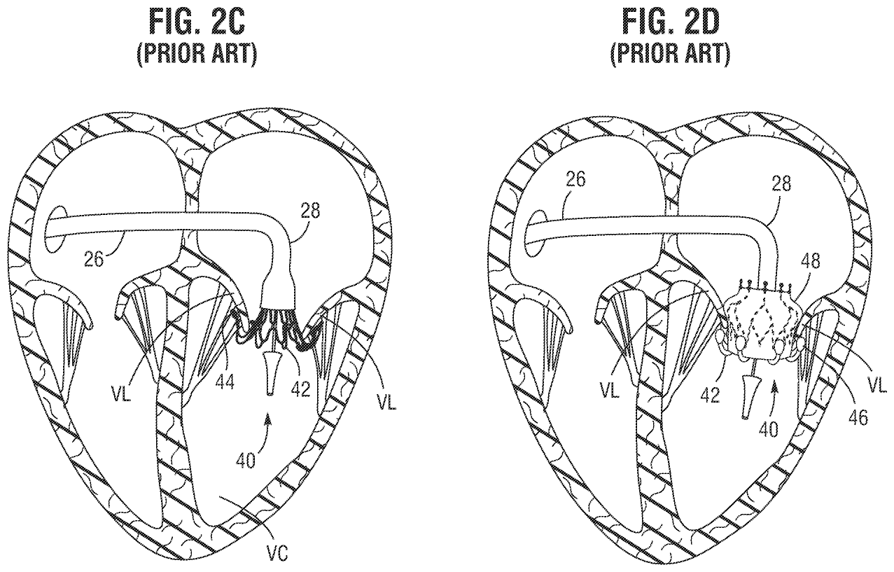

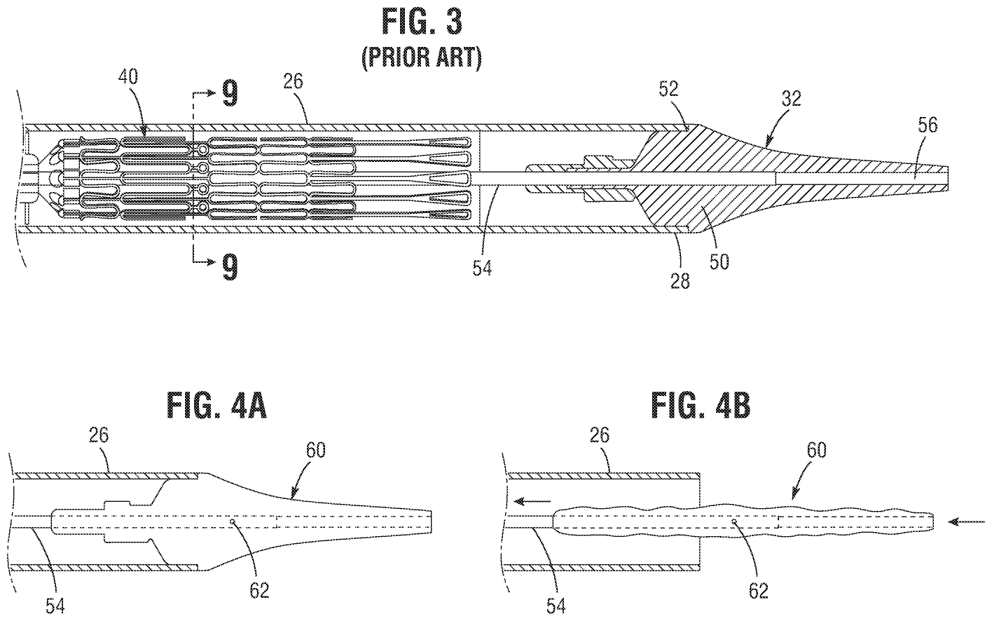

To better understand the structure of the conventional nose cone 32, FIG. 3 schematically illustrates a longitudinal sectional view through a conventional delivery catheter 26. The nose cone 32 has an elongated, tapered front-end which widens to a proximal base portion 50. The base portion 50 is preferably shaped with an exterior step 52 that mates with and projects from the distal end 28 of the delivery catheter 26. In conventional embodiments, the nose cone 32 engages the distal end 28 of the delivery catheter 26 with an interference or friction fit and is further secured on the distal end via attachment to an elongated guidewire tube 54. Although not shown, the guidewire tube 54 typically extends proximally through the entire delivery system 20 to the proximal handle 22. The guidewire tube 54 is hollow for passage of a guidewire, not shown here for the sake of clarity, and is fixedly secured within a through bore 56 formed within the nose cone 32 for continued passage of a guidewire therethrough. A collapsed prosthetic valve 40 is seen within the delivery catheter 26 just proximal to the nose cone 32. For better understanding, the discussion of various alternative nose cones below will use some of the same element numbering for the various features of the valve delivery system as needed for explanation.

One embodiment of an alternative nose cone 60, shown in FIGS. 4A and 4B, is collapsible to a collapsed state that does not project distally from the distal end of the delivery catheter 26. For procedures on the tricuspid annulus, as seen in FIG. 1A, a tapered nose cone or tip is not a critical feature since the catheter 26 does not cross a puncture in the atrial septum. In the example illustrated in FIG. 4A, the nose cone 60 is an inflatable element which, when inflated, assumes an identical or similar shape as the conventional nose cone 32. As such, the inflated nose cone 60 engages the end of the delivery catheter 26 as before and projects from the distal end 28 thereof. In one embodiment, the guidewire tube 54 may incorporate a small inflation port 62 for inflating and deflating the nose cone 60. This arrangement may require a plug or seal within the lumen distal to the port 62, several embodiments of which are described below.

The inflated nose cone 60 performs essentially the same function as the conventional nose cone 32 during delivery of the prosthetic heart valve 40 to the annulus. At a certain point, the advantages of the nose cone 60 are realized and its presence becomes a hindrance to further manipulation of the delivery catheter 26. For example, once the delivery catheter 26 has been advanced into proximity with or within the target annulus, the nose cone 60 is not required and it becomes an impediment due to its length and sharp end.

At that time, the alternative nose cone 60 may be deflated, as seen in FIG. 4B, such as by withdrawing the inflation fluid into the guidewire tube through the port 62. Once deflated, and thus radially collapsed, the nose cone 60 may be retracted within the delivery catheter 26 by pulling on the guidewire tube 54, as indicated in FIG. 4B. Depending on size constraints, the deflated nose cone 60 may be retracted proximally through the compressed heart valve within the catheter 26, or the nose cone 60 may be temporarily advanced beyond the distal tip of the catheter 26 to enable passage of the expandable heart valve or other instruments used in the implantation thereof. A typical central bore within a compressed heart valve may be 0.040 inches in diameter, which is small but potentially large enough for proximal nose cone 60 retraction through the valve. Or, the deflated nose cone 60 is displaced distally during the valve implant procedure, and it's reduced size and flaccid state render it essentially out of the way. It should be understood that either of these two steps can be done for a number of the collapsible and removable nose cones described herein.

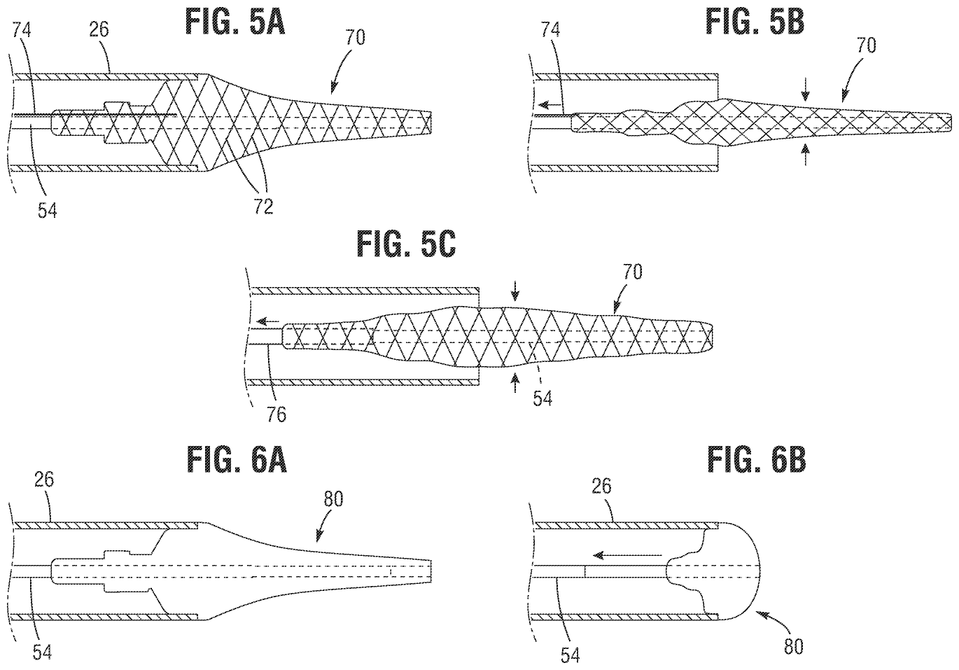

FIGS. 5A-5C illustrate a further alternative nose cone 70 which is also collapsible to a collapsed state that does not project distally from the distal end of the delivery catheter 26. Specifically, the nose cone 70 comprises a plurality of interwoven struts 72 form so that in an expanded configuration, as in FIG. 5A, the nose cone 70 has the same or similar shape as a conventional nose cone 32. Once again, the expanded nose cone 70 may be secured on and project from the distal end of the delivery catheter 26 and be separately manipulated via a guidewire tube 54.

When the nose cone 70 is no longer required, it may be radially collapsed as indicated in FIGS. 5B and 5C. For example, a pull wire 74 may be connected to a portion of the collapsible structure of the struts 72 in a manner which facilitates collapse of the nose cone 70 when it is pulled. Another alternative shown in FIG. 5C is to mount a proximal end of the nose cone 70 on a concentric tube 76 slidable over and relative to the guidewire tube 54, while the distal end of the nose cone is mounted to the guidewire tube. Relatively displacing or rotating the telescoped tubes 54, 76 causes collapse of the nose cone 70. Once the nose cone 70 has collapsed, it may be retracted within the delivery catheter 26 to free up space in front of the delivery system.

FIGS. 6A and 6B illustrate a collapsible/invertible nose cone 80, which is collapsible to a collapsed state wherein it has limited or no projection from the distal end of the delivery catheter 26. As before, the nose cone 80 has an expanded configuration which sits on and projects distally from the distal end of the delivery catheter 26. The nose cone 80 collapses upon proximal retraction of the guidewire tube 54 (or a separate pull wire (not shown) extending through the tube 54. The guidewire tube 54 (or pull wire) extends to the distal end of the nose cone 80 which pulls the distal end backward into the larger body of the nose cone. This transition between expanded and collapsed configurations may be visualized as something like inverting an expanded umbrella, or rubber toilet plunger. That is, the nose cone 80 may be formed of an elastomeric material which easily inverts upon itself as in FIG. 6B. Once again, when inverted/collapsed, the nose cone 80 either simply becomes flat or rounded and may be advanced out-of-the-way, or may be retracted within the delivery catheter 26 proximal to the heart valve.

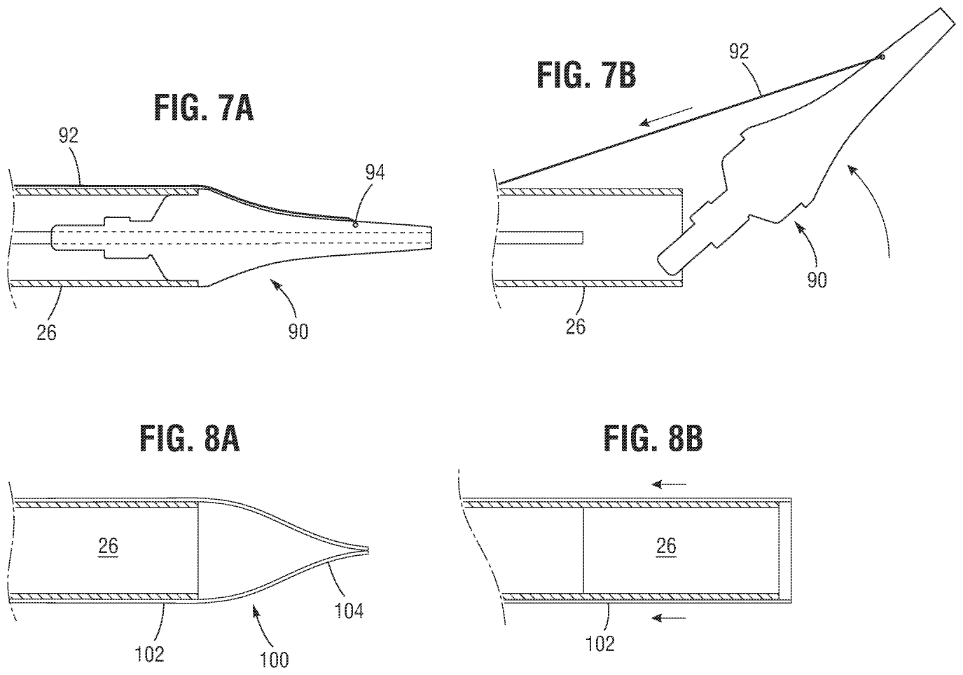

With reference to FIGS. 7A and 7B, a removable nose cone 90 is disclosed. In particular, the nose cone 90 may be configured similar to the conventional nose cone, and sits on the distal end of the delivery catheter 26 with a simple interference fit. A retraction wire 92 extending along the outside of the delivery catheter 26 attaches to an anchor point 94 toward a distal end of the nose cone 90. Because the retraction wire 92 attaches on only one side, pulling the wire in a proximal direction exerts a lateral force on the nose cone 90, as indicated in FIG. 7B. The nose cone 90 can then be removed from the distal end of the delivery catheter 26 and withdrawn from the surgical site. To facilitate removal of the disengaged nose cone 90, it may also be configured to collapse such as described above with respect to FIGS. 4-6.

FIG. 8A illustrates a still further alternative nose cone 100 comprising a retractable sleeve. More particularly, the nose cone 100 is formed by a tubular body 102 terminating at a distal end in a retractable nose 104. For instance, the retractable nose 104 may be formed by flap extensions of the tubular body 102, which come together or coapt much like a duck bill valve. Retracting the tubular body 102 in a proximal direction, such as seen in FIG. 8B, causes the flaps of the retractable nose 104 to separate so that the entire nose cone 100 is pulled in a proximal direction over the delivery catheter 26. The nose cone may be formed with two or more individual flaps or may be formed of a continuous tube shaped with a pointed end.

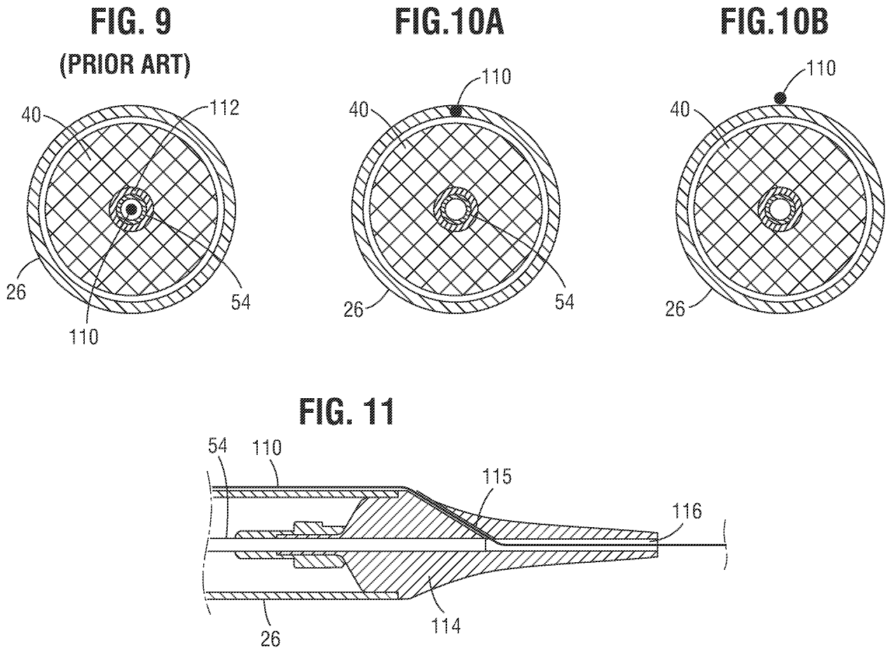

FIG. 9 is a radial sectional view taken along line 9-9 of FIG. 3 through a crimped balloon-expandable prosthetic heart valve 40 held within a valve delivery system. As mentioned above, the delivery systems may advance both self-expandable and balloon-expandable valves, and FIG. 3 may depict either. The valve 40 is radially crimped to fit within the delivery catheter 26. Within a central bore through the valve 40 passes an elongated guidewire tube 54 that receives a guidewire 110. An inflation balloon 112 around the guidewire tube 54 is crimped within the prosthetic valve 40. FIG. 9 shows a conventional placement of the guidewire 110; that is, through the center of the delivery system.

FIGS. 10A and 10B are radial sectional views similar to that of FIG. 9 but where the guidewire 110 extends along alternative pathways that are not centered within the delivery catheter until the distal end of the delivery catheter. For example, the guidewire 110 may be routed through a passage formed in the delivery catheter 26, as seen in FIG. 10A. This leaves the interior of the guidewire tube 54 empty for passage of fluid or supplemental instruments. Alternatively, routing the guidewire 110 along the wall of the delivery catheter 26 enables the guidewire tube 54 to be reduced in diameter, which in turn enables a reduction in the crimped diameter of the prosthetic valve 40 (and balloon 112 if present). Another possible route for the guidewire 110 is completely exterior to the delivery catheter 26, as seen in FIG. 10B. Although not shown, the guidewire 110 may be held within a continuous or intermittent tunnel on the exterior of the delivery catheter 26 to avoid separation therebetween. Again, passing the guidewire 110 along the delivery catheter 26 in a location other than within a central guidewire tube frees up diameter within the delivery catheter 26 for a smaller crimped valve, or to enable the guidewire tube to be used for other purposes. In one variation, the delivery catheter has a guidewire lumen that extends only along a distal portion of the delivery catheter. This arrangement may facilitate setup because it is not necessary to advance the entire delivery catheter over the guidewire.

FIG. 11 is a longitudinal sectional view through an alternative nose cone 114 adapted to provide an angled pathway for a guidewire 110 routed along a passage in or outside a delivery catheter 26, such as seen in FIGS. 10A and 10B. As mentioned, the guidewire 110 extends along the exterior of the delivery catheter 26 until it reaches the nose cone 114, whereupon it angles inward through an angled channel 115 to a central bore 116 of the nose cone. The same terminal pathway for the guidewire 110 may be provided for the embodiment of FIG. 10A, where the guidewire extends through a longitudinal passage in the wall of the delivery catheter 26. Once again, this frees up space within a central tube 54 for other uses, or the tube 54 may be reduced in size which enables a reduction in size of the entire delivery catheter 26.

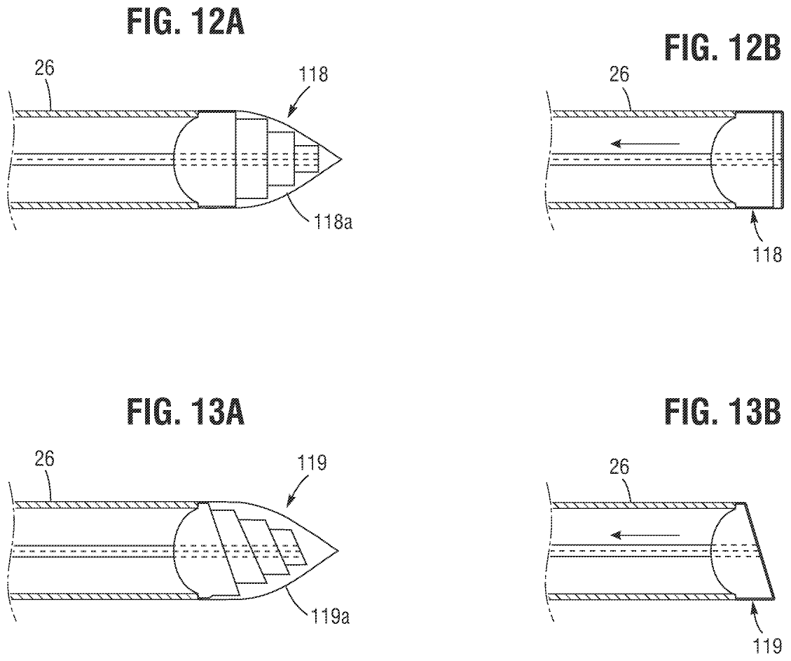

FIGS. 12A and 12B are schematic views of the distal end of a delivery catheter 26 showing a further alternative nose cone 118 in extended and collapsed states. As explained, for procedures on the tricuspid annulus, as seen in FIG. 1A, a tapered nose cone or tip is typically not required since the catheter 26 does not cross a puncture in the atrial septum. While a tapered tip would aid insertion of the delivery catheter 26 into the groin (i.e., to access the femoral vein), it would present too large of an obstacle once the delivery system to enter the right ventricle through the tricuspid valve.

As a solution to this dilemma, a collapsible nose cone 118 surrounded by a flexible cover 118a may be provided on the distal end of the delivery catheter 26. The nose cone 118 may be configured with a series of connected and nested layers in a “layer-cake” stack that are biased or temporarily held into the tapered shape shown in FIG. 12A, but which can be collapsed upon themselves by retracting a pull wire or inner tube, for example, as shown in FIG. 12B. For instance, the inner tube 54 shown in FIGS. 6A and 6B may be used. The smaller layers nest within the larger ones, and the flexible cover 118a retracts along with the nose cone 118. After collapse, the nose cone 118 may be temporarily advanced beyond the distal tip of the catheter 26 to enable passage of an expandable heart valve or other instruments used in the implantation thereof.

FIGS. 13A and 13B show the distal end of the delivery catheter 26 and a still further collapsible nose cone 119 in both extended and collapsed states. In this embodiment, the nose cone 119 has layers that are nested in an angular manner and that may collapse by rotating or retracting a pull wire, as seen in FIG. 13B. Once again, a flexible cover 119a provides a smooth tapered exterior to facilitate introduction into the groin area and femoral vein, for example. After collapse, the nose cone 119 may also be temporarily advanced beyond the distal tip of the catheter 26 to enable passage of an expandable heart valve or other instruments used in the implantation thereof.

### Inflatable Systems

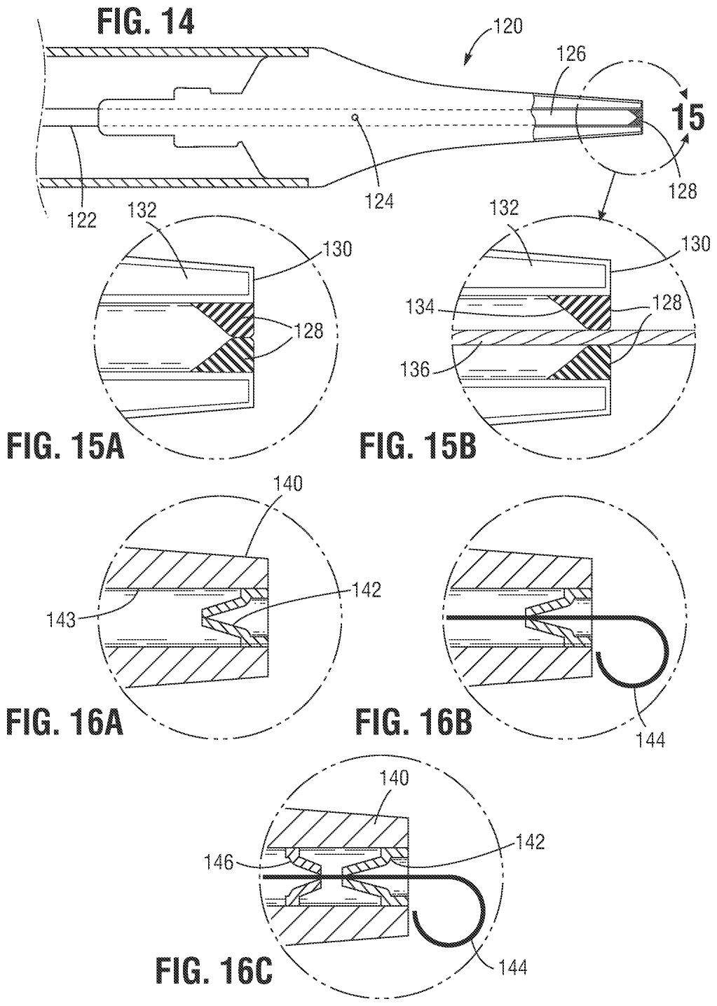

FIG. 14 is a longitudinal sectional view through an inflatable nose cone 120 held on the end of a central tube 122, which may be a guidewire tube. The central tube 122 continues through the middle of the nose cone 120 and has one or more side ports 124 open to an inner lumen 126. A distal seal 128 provides a fluid closure at a distal end of the lumen 126.

FIGS. 15A and 15B are enlargements of a distal end of the inflatable nose cone 120 illustrating alternative uses of the distal seal 128. FIG. 15A illustrates what may be referred to as a zero seal, which means an annular seal 128 which closes upon itself to seal the distal end of the lumen 126 when there are no instruments through the seal. The presence of a zero seal 128 enables the lumen 126 of the central tube 122 to be pressurized with insufflation fluid for use in a variety of contexts. For instance, the nose cone 120 itself may be inflatable, and be formed by an outer wall 130 surrounding an inner inflation space 132. Because the nose cone 120 in the illustrated embodiment surrounds the central tube 122, the inner inflation space 132 is circular in radial section.

FIG. 15B illustrates a guidewire 136 which may be passed through the annular zero seal 128. The seal 128 is preferably elastomeric and flexes outward upon passage of the guidewire 136, but provides a good fluid seal therearound for continued use of the lumen 126 for pressurization. The zero seal 128 is shown with a conical inner wall 134 which facilitates passage of the guidewire 136.

FIG. 16A is an enlargement of the distal end of a nose cone 140 showing an alternative distal seal 142 positioned at the distal end of an inner lumen 143, and FIG. 16B shows passage of a guidewire 144 through the seal. The nose cone 140 is preferably solid, but may also be inflatable as described above with respect to FIGS. 14-15. The distal seal 142 is formed as a duck bill-type seal, with two elastomeric flaps 145 which come together along a central axis of the inner lumen 143 and are angled in a proximal direction. Pressure within the lumen 143 thus tends to close the two elastomeric flaps 145, thus better enabling pressurization of the inner lumen 143. FIG. 16C shows the addition of a lead-in seal 146 proximally from the duck bill seal 142 and facilitates passage of the guidewire 144 through the duck bill seal. That is, the lead-in seal 146 is conical and tapered toward a central axis so that the guidewire 144 is channeled between the flaps 145 of the duck bill seal 142.

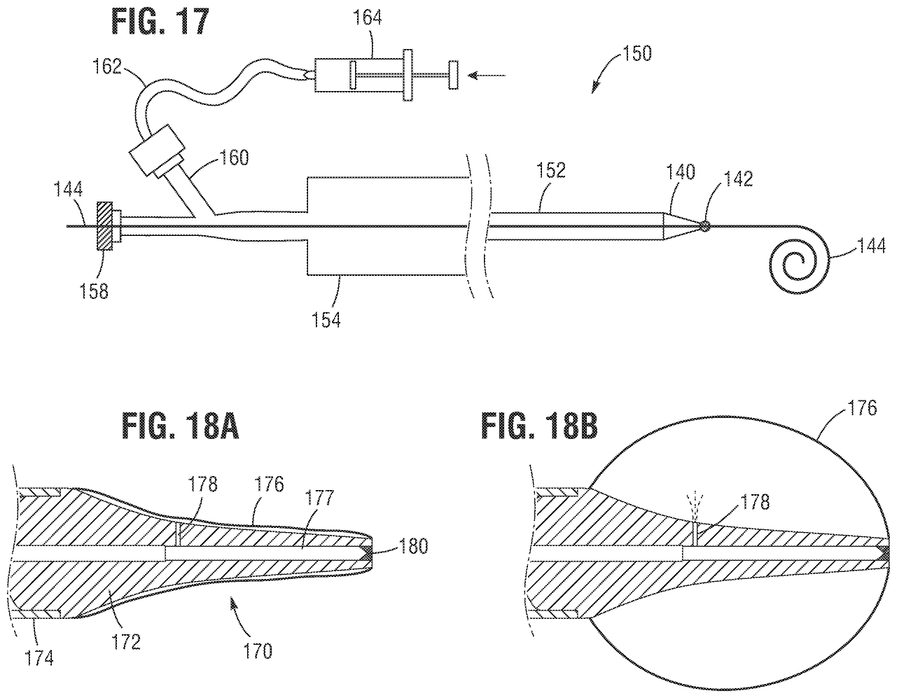

FIG. 17 is a schematic illustration of a system 150 for providing fluid to a valve delivery guidewire tube that doubles as an inflation tube, as has been described herein. The valve delivery system may incorporate the nose cone 140 having the distal seal 142 as described with reference to FIGS. 16A-16C. An elongated guidewire 144 extends through the entire valve delivery system, which includes a delivery catheter 152 attached to a proximal handle 154. The guidewire 154 passes through a proximal seal 158 on the handle 154, and then extends the length of the system through the distal seal 142. An inner lumen within the delivery catheter 152, such as the inner lumen 143 described above, can then be pressurized with inflation fluid. For example, an angled side port 160 off the proximal handle 154 may be in fluid communication with the inner inflation lumen and also be in fluid communication with a flexible hose 162 attached to a source of pressurizing fluid, such as a manual syringe 164. Pressurizing fluid may be saline, for example, such that the inner inflation lumen can be supplied with pressurized saline as desired.

FIGS. 18A and 18B are longitudinal sectional views of a still further nose cone 170 having a solid body 172 that fits on the distal end of a delivery catheter 174. An outer balloon 176 surrounding the solid body 172 may be inflated and deflated via an inner inflation lumen 177 that opens to a side port 178. A distal seal 180 provided at the distal end of the inflation lumen 177 enables pressurization thereof. The inflated balloon 176 may be useful for navigating through tangled and sometimes fragile anatomy, such as through a native heart valve and into a ventricle having chordae tendineae.

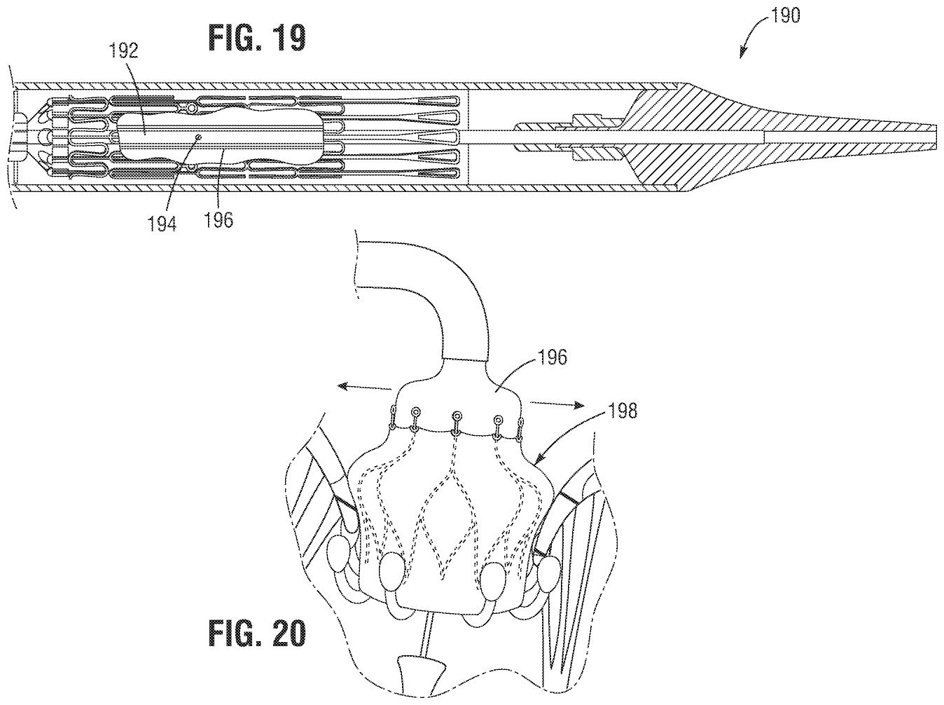

FIG. 19 is a longitudinal sectional view through the distal section of a valve delivery system 190 shown to illustrate one possible usage of a dual guidewire/inflation tube 192. A portion of a crimped valve has been removed to illustrate the dual guidewire/inflation tube 192 having a one or more side ports 194 form therein. A valve expansion balloon 196 surrounds the tube 192 and has an interior space open to the one or more side ports 194. FIG. 20 illustrates expansion of the balloon 196 within an expandable prosthetic heart valve 198, such as one similar to that shown being implanted in the sequence of FIGS. 2A-2C.

### Septal Stabilizer

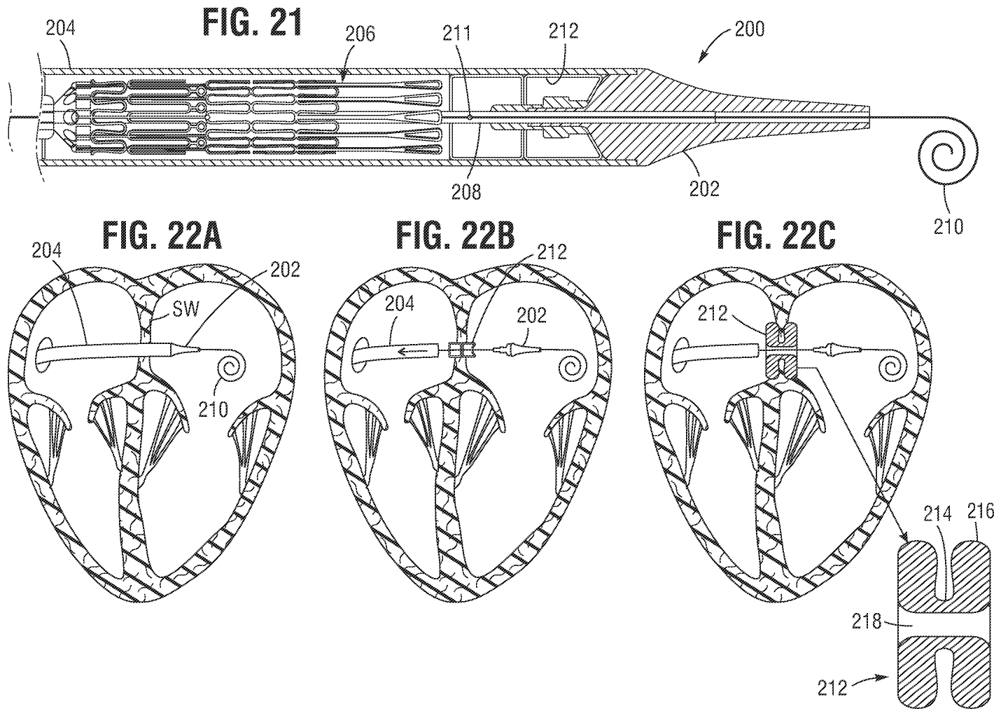

FIG. 21 is a longitudinal sectional view through the distal section of a valve delivery system 200 designed to facilitate passage through a septal wall within the heart. The system 200 has a distal nose cone 202 attached to a distal end of a delivery catheter 204. A crimped prosthetic heart valve 206 resides within the catheter 204. An inner tube, such as an inner guidewire tube 208 extends the length of the system and through the valve 206 to attach within the nose cone 202, and a guidewire 210 may be passed therethrough. The inner guidewire tube 208 has one or more side ports 211 open to an interior space of a septal stabilizing balloon 212.

FIGS. 22A-22C are cross-sections of a heart showing a sequence of steps in a deploying the septal stabilizing balloon 212 of FIG. 21 during a mitral valve replacement procedure. A puncture will be made in the septal wall SW through which a guidewire sheath (not shown) and then the guidewire 210 will be introduced. Once the guidewire 210 is through the septal wall SW, the guidewire sheath is removed, and the distal end assumes the atraumatic coiled shape as shown. Subsequently, the delivery catheter 204 is advanced through the vascular and along the guidewire 210 until the nose cone 202 has crossed the septal wall SW.

At this point, as seen in FIG. 22B, the nose cone 202 is held stationary and the catheter 204 is retracted. The septal stabilizing balloon 212 remains in place within the puncture through the septal wall SW. Positioning the balloon 212 within the puncture through the septal wall SW may be assisted by the use of fluoroscopy or other such visualization means. Subsequently, the septal stabilizing balloon 212 is inflated into the shape shown in FIG. 22C. Namely, the septal balloon 212 defines a spool or hourglass shape with a central circular groove 214 that receives the septal wall SW and a pair of annular lobes 216 that flank the septal wall SW.

A central through bore 218 provides a passageway for subsequent advance of the delivery catheter 204, and replacement of the mitral valve. The septal stabilizing balloon 212 thus provides a barrier between the delivery system including the catheter 204 and the septal anatomy so as to distribute the insertion in steering loads to a larger surface area and reduce risk of concentrated localized force and pinching. Moreover, the balloon 212 creates a support for the delivery system by stiffening up the septal wall SW during the valve replacement procedure.

### Guidewire Stiffening

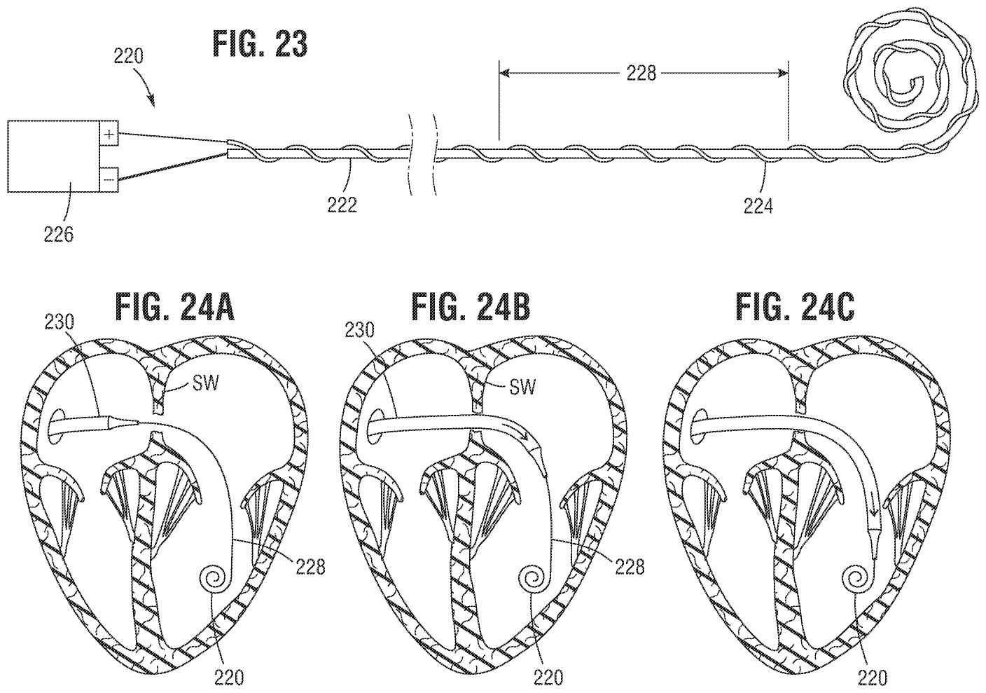

FIG. 23 illustrates an exemplary convertible guidewire 220 having a portion that may be stiffened when desired. Typically, guide wires are made of stainless steel and are relatively flexible. In heart valve replacement procedures, however, conventional flexible guide wires may be insufficient to guide the delivery catheter into position across the native annulus, such as, for atrioventricular valve replacement procedures as described herein. More specifically, as the delivery catheter advances over a flexible guidewire which has been positioned within the ventricle, the stiffness of the catheter and associated components overcomes the minimal stiffness of the guidewire and tends to pull the guidewire out of position.

As a proposed solution, FIG. 23 shows a convertible guidewire 220 having an inner core wire 222 surrounded by a coil wire 224 that extends along its length. Both the core wire 222 and the coil wire 224 are electrically conductive, and are placed in electrical communication at their distal ends. The coil wire 224 has an insulating coating on its exterior to avoid shorting the circuit out. An electrical source 226 is schematically shown providing current to the circuit.

The core wire 222 has a bimetal makeup; namely it is made of Nitinol with sections of differing Austenite finish temperatures (Af) to create discrete sections of stiffness in the guidewire when desired. For the purpose of definition, Austenite is the high temperature parent phase of the Nitinol alloy having a B2 crystal structure, while Martensite is the lowest temperature phase in Nitinol shape memory alloys with a B19′ (B19 prime) monoclinic crystal structure. The Austenite finish temperature (Af) is the temperature at which Martensite (or R-phase) to Austenite transformation is completed on heating of the alloy. Nitinol remains highly flexible in the Martensitic phase and then reverts to a memory shape or becomes stiff when it transitions to an Austenitic phase.

In the illustrated embodiment, the core wire 222 is treated to have at least one section 228 of different Af temperature by virtue of heat setting the wire differently in different zones. Specifically, section 228 is heat treated to have a higher Af temperature than the remainder of the core 222. Due to high core Af temperature, wire in section 228 is flexible at or below body temperature (NiTi core is shape memory/martensite). When desired, the core wire section 228 transforms to a stiff member via inductive current applied to coil; i.e., NiTi core is super elastic/austenite when current is applied to coil).

In this way, a majority of the core wire 222 may remain flexible at body temperature, while a particular section such as section 228 may be stiffened upon application of electrical inductive current and thus heating of the guidewire 220. In particular, a convertible section 228 near the coil distal end of the guidewire 220 may be selectively stiffened. In one example, the convertible section 228 is heat treated so that its Af temperature is greater than body temperature (˜37° C.), such as 60° C.

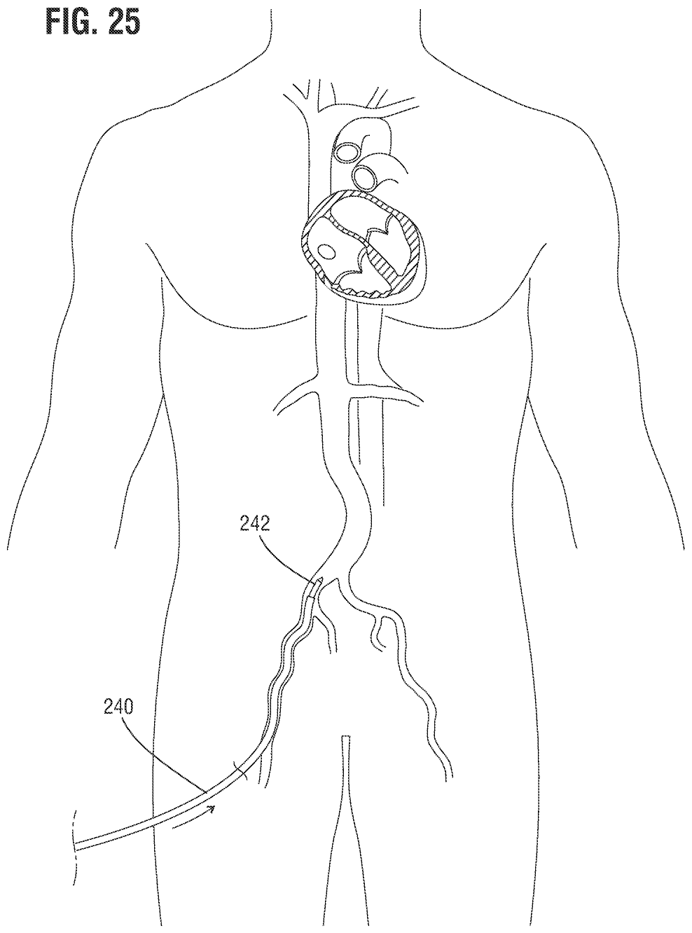

FIGS. 24A-24C illustrate a sequence of usage of the convertible guidewire 220 of FIG. 23. Initially, a puncture will be made in the septal wall SW through which a guidewire sheath (not shown) and then the guidewire 220 will be introduced. The guidewire sheath is directed down through the mitral valve into the left ventricle and then removed so that the distal end of the guidewire 220 assumes the atraumatic coiled shape as shown. At this stage, no current has been applied to the guidewire 220, and it remains quite flexible. If the delivery catheter 230 were advanced across the septal wall SW in an attempt to enter the left ventricle, the stiffness of the catheter would pull the guidewire 220 back up into the left atrium.

Instead, current is applied to the guidewire 220 which stiffens the convertible section 228. This enables advancement of the delivery catheter 230 along the guidewire 220 across the septal wall SW and into the left ventricle, as seen in FIGS. 24B and 24C. Once the delivery catheter 230 has crossed the mitral valve, the current in the guidewire 220 can be removed so that the guidewire assumes its fully flexible properties as before.

### Convertible/Compressible Access Sheaths

FIG. 25 is a schematic representation of a transvascular method of introducing a flexible catheter assembly 240, 242 into the heart to perform procedures, showing a tortuous vasculature pathway which presents challenges to the procedure. The catheter assembly includes a catheter access sheath 240, and a concentric catheter 242 that extends from the distal end of the sheath. The assembly is shown during an initial stage of introduction to the body for a procedure in the heart, with the access sheath 240 inserted through an incision into the venous, and a portion of the catheter 242 visibly extending from the sheath. Often, the venous system from the femoral vein up toward the ascending aorta is relatively tortuous as shown, and requires great flexibility in the catheter assembly 240, 242. However, both the access sheath 240 and the extensible catheter 242 require a minimum amount of axial stiffness to be enable the surgeon to advance the components through the vasculature. The conflict between flexibility and stiffness creates trade-offs.

Moreover, delivery system profiles for transcatheter mitral and tricuspid replacement catheters 242 require large IDs (>30 Fr or 10 mm). This can pose challenges for access, especially if an additional sheath 240 is required to gain access, thereby adding additional profile on top of the delivery system, pushing >33 Fr (11 mm) and above. There is currently no large bore sheath 240 available to gain access for devices above 26 Fr (the GORE® DrySeal Flex Introducer Sheath is the largest known commercially available sheath with a max ID of 26 Fr). Thus, a low-profile sheath solution for access would be very beneficial.

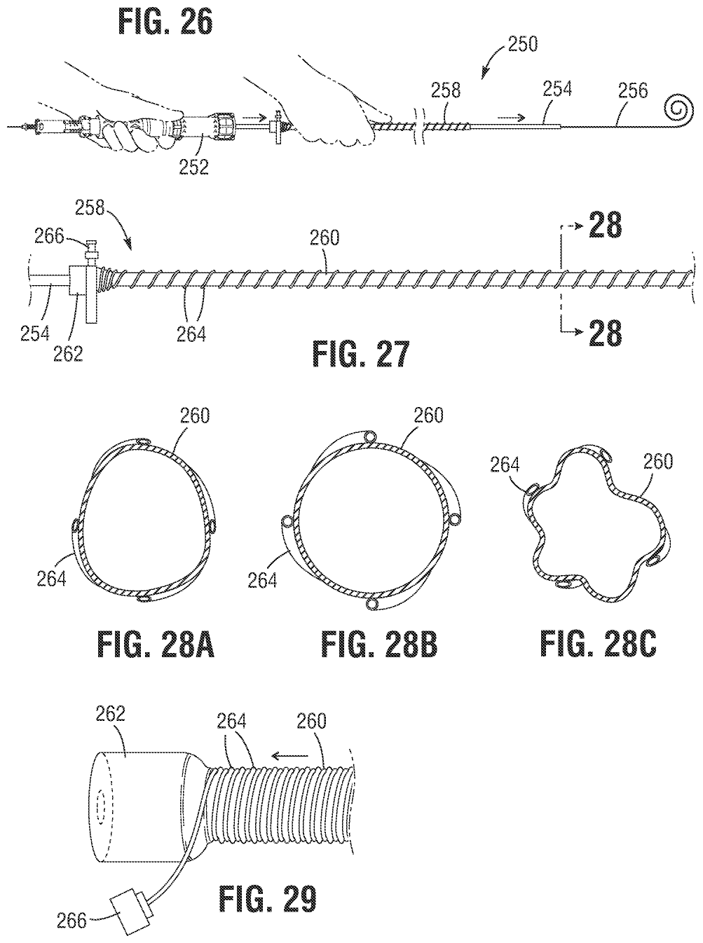

FIG. 26 is a view of an entire valve delivery system 250 illustrating relative movement between a control handle 252 and inner catheter 254 riding over a guidewire 256 with respect to an access sheath 258. In one typical operation, the surgeon or technician holds a portion of the access sheath 258 stable while distally advancing the control handle 252 and catheter 254. If the access sheath 258 is bent in multiple places because it conforms to a tortuous anatomy, advancing the catheter 254 may be difficult. However, the access sheath 258 must have a certain amount of flexibility to navigate the tortuous anatomy. The access sheath 258 is thus configured to be converted from a more flexible to a stiffer configuration, as will be described.

FIG. 27 illustrates the convertible access sheath 258, as would be used in the system of FIG. 26 having an inflatable stiffening spiral thereon. More particularly, the access sheath 258 comprises an elongated flexible tube 260 extending distally from a proximal hub 262. The hub 262 preferably has one or more valves to seal around the inner catheter 254 which slides through the tube 260. A narrow inflatable filament 264 is spirally arranged around the elongated tube 260 from the hub 262 along at least a majority of the tube, and potentially the entire length of the tube. There may be one or multiple filaments 264 spirally surrounding the tube 260.

Although not shown, an exterior tubular cover may be provided around the filament(s) 264 to maintain a smooth outer surface for the sheath 258. A fill valve 266 which may be provided on the hub 262 supplies insufflation fluid (saline, air, or any fluid medium) to the inflatable filament 264.

FIGS. 28A-28C are radial sectional views showing alternative configurations of the convertible access sheath 258. In FIG. 28A, the elongated tube 260 is shown having a typical cross-sectional shape as seen in the body when the filaments 264 are deflated. That is, the tube 260 has enough radial integrity to remain somewhat circular, and the entire access sheath 258 remains relatively flexible for passage through a tortuous anatomy. To the contrary, FIG. 28B shows the filaments 264 inflated with fluid, which tends to stiffen the entire sheath 258 and helps maintain the tube 260 in a circular cross-sectional shape. Thus, the access sheath 258 can alternately be made more or less flexible depending on whether the filaments 264 are filled with fluid or not. FIG. 28C are illustrates a slightly modified version of the access sheath 258 wherein the flexible tube 260 is formed with pleats or longitudinal folds so that it is somewhat radially collapsible. Subsequently, injecting fluid into the filaments 264 expands the tube 260 into a circular configuration, as seen in FIG. 28B.

Typically, sheath support structures are metal coils and/or braids to maintain hoop strength and provide resistance to kink, but these are static (built to one diameter) and still have tendencies to kink/buckle. An inflatable support structure which is the filaments 264 can provide a transient support when needed, and then deflated when not. The advantage here is that when deflated, it can take a smaller profile shape upon introduction and then be inflated to its intended diameter to allow passage of the catheter 242. Additionally, since the sheath 258 is non-metallic it can be deflated and peeled away (or “scrunched” back), such as if the sheath is only desired for access and the physician wants to remove it. This has advantages if you want to provide a long access sheath to get through particularly tortuous veins and pulled back over the device for the rest of the procedure.

For instance, FIG. 29 is a schematic view of a proximal end of the convertible access sheath 258 illustrating the capacity for axial compression. That is, when the filaments 264 are deflated, the entire sheath 58 may be axially compressed in an accordion-like fashion. Such a result may be facilitated by providing circumferential pleats or other such collapsible structure within the tube 260.

Another problem with access sheaths using metallic coils or braids is that the sheets are generally fixed in length. It may be desirable to utilize a long sheath to get past certain anatomical landmarks, but the sheath is only needed for catheter introduction. With a long sheath, the surgeon may not be able to withdraw the sheath completely, which may inhibit the movement of the components of the system.

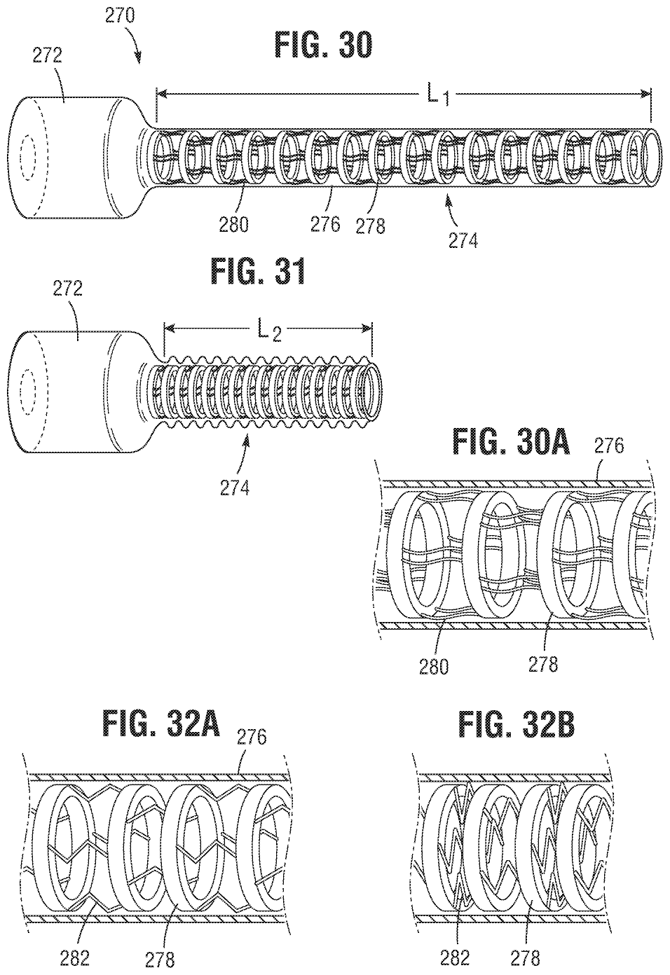

FIG. 30 is an elevational view of an alternative compressible access device 270 in an extended configuration, and FIG. 30A is an enlargement of a portion thereof. The sheath 270 may consist of a proximal hub 272 having an elongated sheath 274 extending distally therefrom. The wall construction of the elongated sheath 274 enables axial compression from an extended configuration to an axially collapsed configuration. Specifically, the elongated sheath 274 may be formed by inner liner, an axially compressible support structure, and an outer jacket for the introducer sheath. In the illustrated embodiment, the sheath 274 comprises an outer jacket 276 surrounding a series of axially spaced apart rings 278 that are joined together by axially compressible struts 280. The axially compressible support structure comprising the rings 278 joined by the struts 280 may be formed by a laser cut hypotube pattern that has a series of compressible sections. The struts 280 are shown with a serpentine configuration that enables axial collapse. Rather than a continuous coil, this describes a specific pattern that allows for the compression of inner struts 280 that link the radial support rings 278. In the illustrated embodiment, there are four pairs of struts 280 extending between adjacent rings 278, and the pairs of struts between two rings are rotationally offset from the pairs of struts between the next two rings. Of course, the number and arrangement of struts 280 can be very. Although the entire sheath 274 is shown constructed in a compressible manner, the compressible portion be limited to just a section thereof, such as a middle section.

FIG. 31 is an elevational view of the compressible access sheath of FIG. 30 showing axial compression thereof. In particular, an extended length L₁ as seen in FIG. 30 may be reduced to a constricted length L₂ as seen in FIG. 31. The constricted length L₂ may be between 30-70% of the extended length L₁.

Finally, FIGS. 32A and 32B are enlargements of several links in an alternative compressible access sheath having a wall construction that enables extended and axially compressed configurations, respectively. As before, the sheath comprises an outer jacket 276 surrounding a series of axially spaced apart rings 278 joined by axially compressible struts 282. In this embodiment, the struts 282 are formed in a zigzag configuration rather than serpentine. Between each two rings 278 are for individual struts 282 distributed 90 degrees angularly. Again, the four struts 282 between two of the rings 278 may be rotationally offset with respect to the four struts between the next two rings.

### Removable Access Sheath Tip

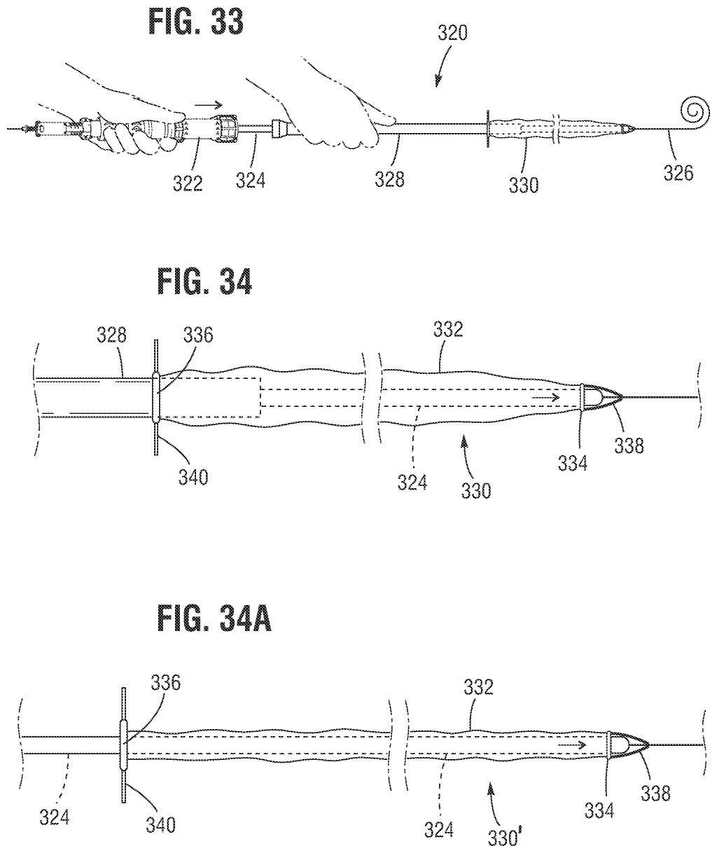

FIG. 33 is a view of an entire valve delivery system 320 illustrating forward movement of a control handle 322 and inner catheter 324 along with an access sheath 328 over a guidewire 326. In one typical operation, the surgeon or technician holds a portion of the access sheath 328 stable while distally advancing the control handle 322 and catheter 324. Prior to distal displacement of the catheter 324, and implantation of a prosthetic valve carried therein, the access sheath 328 is inserted into the patient's vasculature. While positioning the delivery system 320 for tricuspid valve procedures, there needs to be sufficient clearance between a distal tip thereof and the right ventricle to ensure proper position, especially depth control. Although a tapered tip on the distal end of the delivery catheter 324 is beneficial for gaining access to the vasculature, such a tapered tip reduces maneuvering room within the right ventricle.

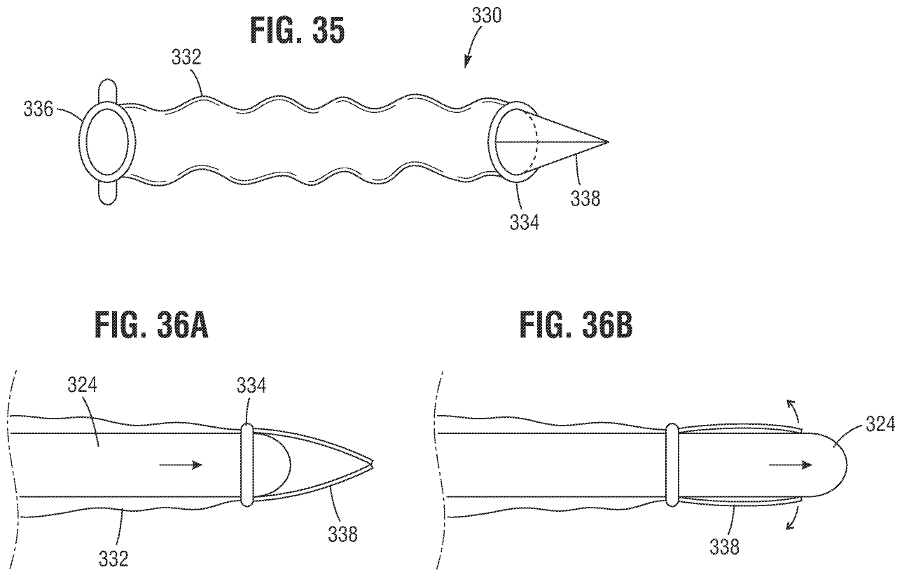

Consequently, FIGS. 33 and 34 illustrate a distal end of the access sheath 328 with a break-away delivery system tip 330 incorporated thereon. FIG. 35 shows the break-away delivery system tip 330, and FIGS. 36A-36B show distal displacement of the delivery catheter 324 therethrough. The break-away tip 330 comprises a flexible tubular bag or “shaft” 332 having O-ring style seals 334, 336 on distal and proximal ends. The distal and proximal seals 334, 336 are sized to provide hemostatic barriers around the access sheath 328 and delivery catheter 324. A tapered distal end 338 extends around a distal end of the delivery catheter 324 and provides an atraumatic forward end for introduction into and subsequently through the vasculature to the tricuspid annulus. The tapered distal end 338 may be formed by a pair of leaves or petals which come together at a point but which can be flexed apart upon passage of the delivery catheter 324. The leaves or petals of the distal end 338 have sufficient stiffness to provide an atraumatic tapered tip for entry of the delivery catheter 324 through hemostatic seals within the access sheath 328 and/or an incision into the body.

FIG. 34A shows an alternative where the break-away delivery system tip 330′ forms the access sheath itself. That is, a flexible tubular bag or “shaft” 332 having O-ring style seals 334, 336 on distal and proximal ends fits directly around the catheter 324 and provides a hemostatic sheath when introducing the catheter into the body. The delivery catheter 324 and the delivery system tip 330′ for the delivery system 320. The distal seal 334 and tapered distal end 338 are carried along by the catheter 324 as it advances through the incision, thus providing atraumatic entry. At the point where the flexible access bag 332 extends to its full length, and potentially when a pair of outer wings or flanges 340 contact the exterior of the patient's access site, movement of the access bag 332 is stopped. The tip of the catheter 324 then pushes through and beyond the distal end 338 petals and the heart valve delivery and deployment can proceed without the extended tip 338.

FIG. 36A shows distal advancement of the delivery catheter 324. Initially, the frictional fit between the distal seal 334 and the delivery catheter 324 carries the tapered distal end 338 forward with the catheter. The surrounding flexible shaft 332 provides a hemostatic barrier around the delivery system 320, be it the access sheath 328 and catheter 324, or just the catheter 324 as in FIG. 34A. The entire length of the break-away tip 330 is less than the length needed to advance the delivery system 320 to the tricuspid annulus, and at some point a pair of outer wings or flanges 340 halt further advancement of the tip 330. The flanges 340, which could also comprise an annular flange, contact the exterior of the patient's access site to halt movement of the tip 330. Subsequently, FIG. 36B shows continued distal advancement of the delivery catheter 324 through the flexible leaves or petals of the distal end 338. This removes the tapered end from the access sheath 328 prior to reaching the tricuspid annulus, which thus reduces the distal end profile of the delivery system and improves maneuverability. The distal seal 334 maintains hemostasis around the delivery catheter 324.

The break-away tip 330 is manufactured using known biocompatible materials. Movement of the delivery catheter 324 through the leaves or petals of the distal end 338 is entirely passive, merely depending on the relative lengths of the tip and sheath. The leaves or petals of the distal end 338 may have sufficient stiffness to provide the tapered entry tip, but have enough flexibility to enable the delivery catheter 324 to flex them apart. Alternatively, the leaves or petals may be hinged at their proximal ends to the O-ring seal 334. The material of the tubular bag or shaft 332 may be similar to flexible liners used in protecting surgical incision sites.

### Steerable Catheters

Multi-planar movement of catheters through multiple pull-wires enables users to steer catheters through tortuous vasculature and maneuver into proper implant position. Often when activating a multi-function catheter system, such as used herein to deliver a heart valve, the activation of a first mechanism changes or alters the performance or direction of a second aspect. For example, when a user activates a secondary flex, it may alter the direction in which a primary flex moves. This requires the user to mentally and/or manually compensate for the new movement, which can be challenging. Consequently, the present application contemplates steerable catheters capable of multi-planar movement through multiple pull-wires that are controlled by motors and controllers that, through sensors in the system, automatically detect the movement of any one mechanism in the catheter (flex, rotation, advancement etc.) and adjust the controls to maintain the desired output of the remaining features.

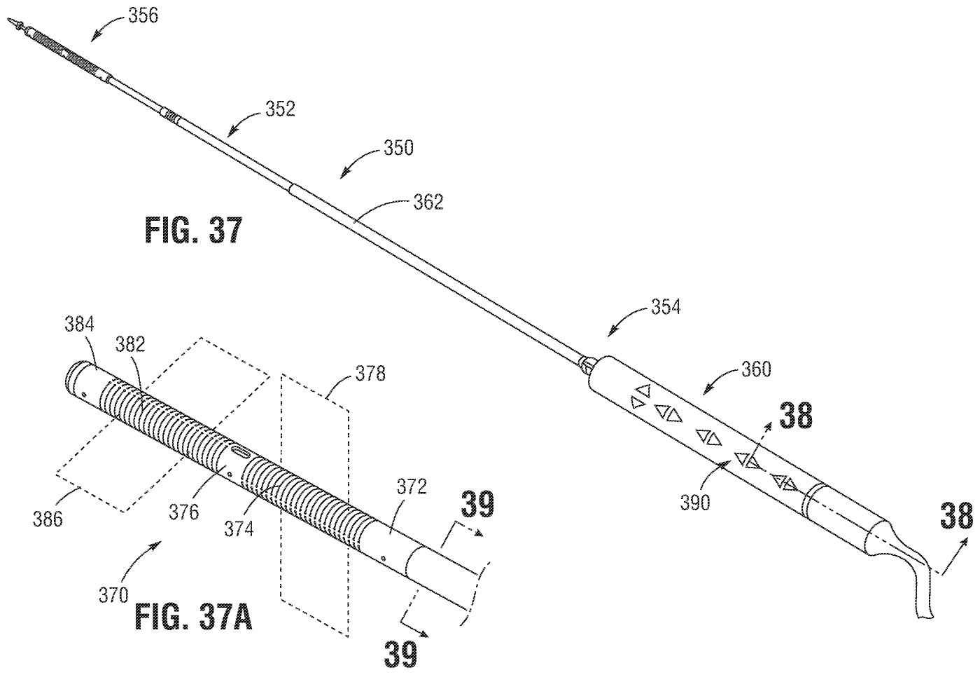

As shown in FIG. 37, an embodiment of a steerable delivery catheter 350 may be used to deploy an implant, such as a prosthetic replacement heart valve, to a location within a patient's body. In some embodiments, the delivery catheter 350 may provide multiple planes (e.g., two or more planes) of deflection for assisting with navigation through a patient's vascular and for enhanced precision during delivery of the implant. While the delivery catheter 350 may be described in certain embodiments in connection with a percutaneous delivery approach, and more specifically a transfemoral delivery approach, it should be understood that features of delivery catheter 350 can be applied to other delivery systems, including delivery systems for a transapical, transatrial, or transjugular delivery approach. The delivery catheter 350 is disclosed in U.S. patent Publication No. 2021/0145576 to Becerra, expressly disclosed herein.

The delivery catheter 350 includes an elongate shaft 352 having a proximal end 354 and a distal end 356, wherein a housing in the form of a handle 360 is coupled to the proximal end. The elongate shaft 352 may be used to hold the implant for advancement of the same through the vasculature to a treatment location. The elongate shaft 352 may further comprise a relatively rigid live-on (or integrated) sheath 362 surrounding an interior portion of the shaft 352 that may reduce unwanted motion of the interior portion of the shaft 352. The live-on sheath 362 can be attached at a proximal end of the shaft 352 proximal to the handle 360, for example at a sheath hub.

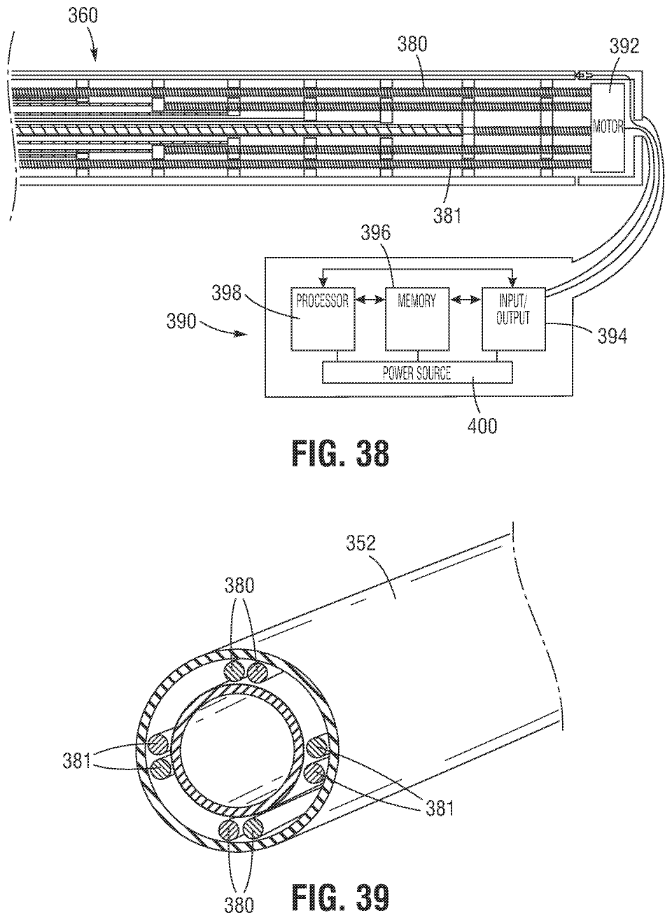

FIG. 37A shows an embodiment of the catheter distal end 356 comprising a rail hypotube 370 (distal end towards the left). The rail hypotube 370 contains a number of circumferential slots and can generally be broken into a number of different sections. At the proximal end is an uncut (or unslotted) hypotube section 372. Moving distally, the next section is a proximal slotted hypotube section 374 which includes a number of circumferential slots cut into the rail hypotube 370. Generally, a series of two diametrically opposed slots are cut around axially spaced circumferential locations forming almost half of the circumference. Accordingly, two diametrically opposed backbones are formed between the slots extending up the length of the hypotube 370, and the slotted hypotube section 374 may be bent within a longitudinal plane 376. The proximal hypotube section 374 can thus be guided by proximal pull wires 380 (see FIGS. 38-39). Moving further distally from the slotted section 374 is an anchor segment 378 where the proximal pull wires 380 connect, and thus slots can be avoided.

Distally following the proximal pull wire anchor segment 378 is a similarly formed distal slotted hypotube section 382. At the distalmost end of the distal slotted hypotube section 382 is a distal pull wire connection area 384 which is again a non-slotted section of the rail hypotube 370. The distal slotted hypotube section 382 can thus be guided by distal pull wires 381 (see FIGS. 38-39). The distal slotted hypotube section 382 is similar to the proximal slotted hypotube section 374, but has more slots cut out in an equivalent length, and thus provides easier bending within a longitudinal plane 386 than the proximal slotted hypotube section 374. In some embodiments, the proximal slotted section 374 can be configured to experience a bend of approximately 90 degrees with a half inch radius whereas the distal slotted section 235 can bend at approximately 180 degrees within a half inch.

The spines of the distal slotted hypotube section 382 are offset from the spines of the proximal slotted hypotube section 374. Accordingly, the two sections will achieve different bend patterns and, in conjunction with axial rotation of the elongate shaft 352, allow for three-dimensional steering of the catheter distal end 356. In some embodiments, the spines can be offset 30, 45, or 90 degrees as shown with bending planes 378, 386, though the particular offset is not limiting. In some embodiments, the proximal slotted hypotube section 374 can include compression coils. This allows for the proximal slotted hypotube section 374 to retain rigidity for specific bending of the distal slotted hypotube section 382.

The handle 360 includes a control device 390 configured to control at least one motor. The control device 390 as shown may include a plurality of control buttons, and may be positioned on the handle 360 as shown or may be located remotely.

FIG. 38 illustrates a cross section of the handle 360 including a motor 392 that may be utilized to actuate pull wires during advancement through the vasculature. The motor may also be used to actuate shafts/sheaths for deploying and releasing the implant at the treatment site.

The control device 390 is configured to control operation of the motor 392 and as seen in FIG. 38 may include an input device and an output device (marked as item 394). The controller 390 may also include a memory 396, a processor 398 and a power source 400. The input device and output device 394 may have a plurality of configurations, including electrical ports or terminals that are configured to transmit electrical signals. The input device may be configured to receive signals from the motor 392 as well as from sensors positioned on the delivery system 350. The output device may be configured to transmit signals to the motor 392 or other components of the system 350 which may be received from the processor 398 or other components of the system 350. In certain embodiments, the input device and output device 394 may comprise wireless transmission devices, such as a Wi-Fi or Bluetooth device or other device configured for wireless communication. In an embodiment in which the controller 390 is positioned remotely from the delivery apparatus, the input device and output device 394 may be configured to transmit and receive information via the Internet or other form of communication medium.

The motor-controlled pull wires 380, 381 thus enable an automated bending solution for the delivery catheter 350. Sensors in the handle 360 and/or shaft 352 may detect flex, rotation, and orientation angle. The processor 398 can then compute a position of the catheter tip and modify or correct the next movement. At a minimum, the processor 398 can adapt to the effect of multiple directional pull wires working at once, and accommodate to result in the proper catheter positioning.

### Positioning Sensors

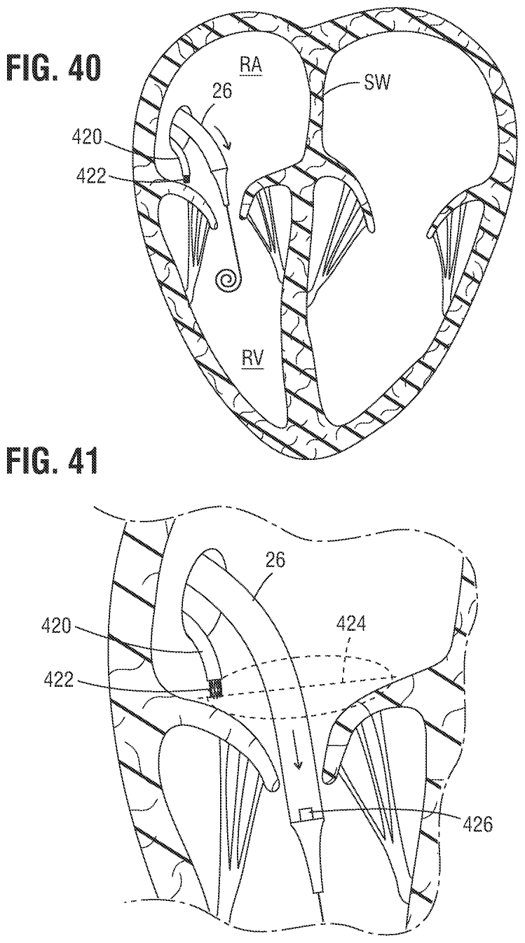

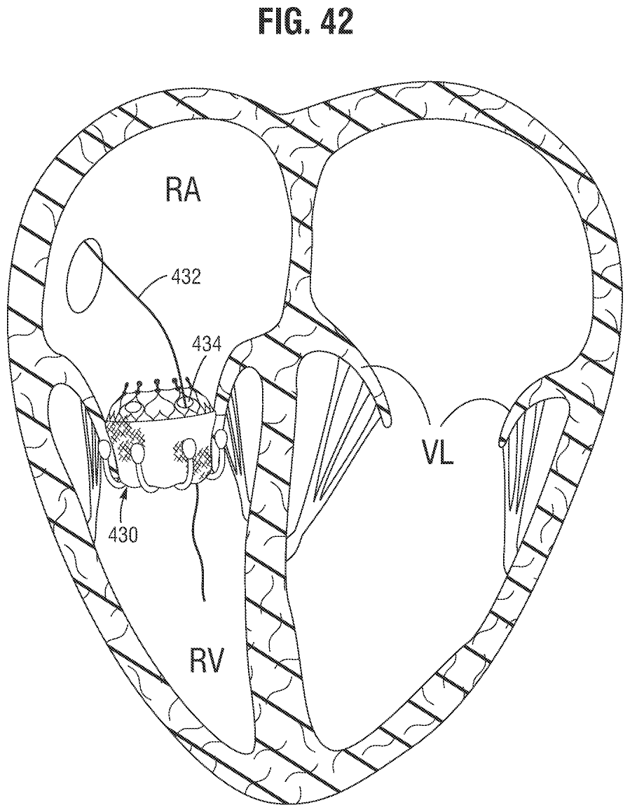

FIGS. 40 and 41 illustrate use of a catheter positioning sensor 420 deployed adjacent to a target tricuspid annulus. During implantation of a prosthetic tricuspid valve, positioning of the delivery catheter 26 within the annulus is critical to ensure that that valve will be in the correct location during deployment. Current technology utilizes echocardiography and fluoroscopy in order to view the delivery system position relative to the annulus, but imaging can be challenging due to patient anatomy and image quality. Poor imaging during the procedure can cause delays and potentially malposition of the delivery system, resulting in a poor implantation.

Consequently, a catheter positioning sensor 420 utilized in concert with the delivery catheter 26 provides a real time datum within the patient's heart at the annulus to provide an accurate reading of the delivery catheter 26 distal tip location to supplement imaging and improve positioning. The catheter positioning sensor 420 comprises a small datum node catheter (2-3 French) inserted alongside the delivery catheter 26 to the tricuspid annulus. The node catheter will follow the inside edge of the patient's right atrium and be positioned at the atrial face of the patient annulus as shown in FIGS. 40 and 41. The distal end could have a single node point emitter 422, or an adjustable ring emitter (shown in dashed line) to better conform to the annulus.

Once in position, the catheter positioning sensor 420 will emit an RF field, or other suitable electromagnetic frequency that would not impact the echo cardiography or fluoroscopy performance, or patient anatomy. The dashed line 424 indicates one such RF field across the atrial side of the tricuspid annulus. On the distal end of the delivery catheter 26, in line with where the valve implant positioning is critical during deployment, a sensor 426 will be positioned to be recognized by the emitter 422. The relative position of the delivery system tip sensor 426 to the emitter 422 will be translated into x-y-z distances and output on a display to the user. The coordinates could also be outputted onto a graphical representation of the patient annulus to help the user visualize the position relative to the anatomy. An additional sensor could be placed on the valve itself, or other sections of the catheter, and be read by the same datum catheter sensor to provide more context and additional measurements to the user on the display.

### Valve Frame Access Ports

Current valve replacement implants pose challenges for future interventions needing to cross the valve. This is particularly challenging with permanent devices such as pacemaker leads which, without providing an alternate pathway, would be forced to be placed through the leaflets of the new prosthetic valve.

Consequently, the present application contemplates implant of a modified prosthetic heart valve 430 as seen in FIG. 42 that accommodates passage of a sensor wire 432, such as a pacemaker lead. Namely, the heart valve 430 has, extending axial therethrough, one or more access ports 434 in the outer body of the valve frame which provide options for further interventions without compromising the integrity of the prosthetic leaflets.

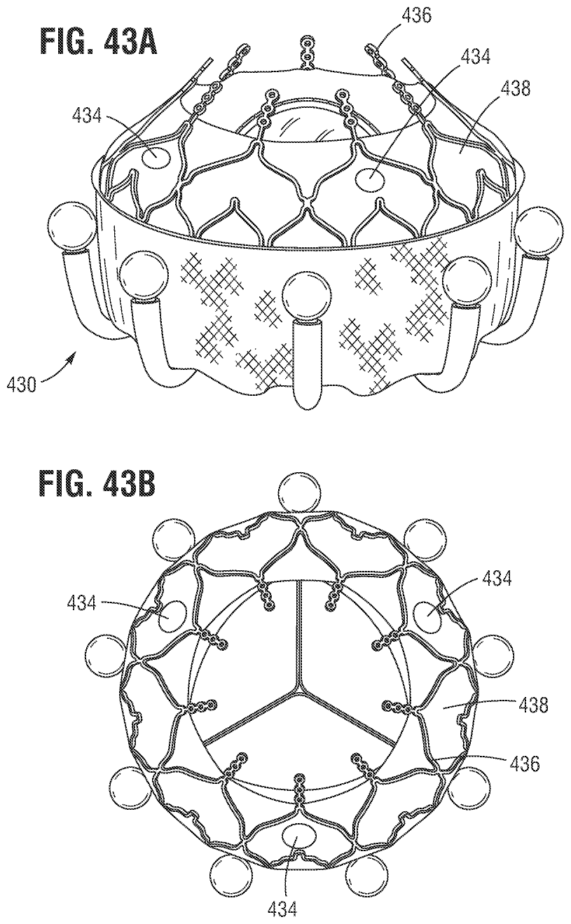

FIGS. 43A and 43B illustrate the modified prosthetic heart valve 430, which in the illustrated embodiment is a modified EVOQUE tricuspid valve made by Edwards Lifesciences Corp. of Irvine, CA, and currently in clinical trials. Among other features, the heart valve 430 has an outer structural frame 436 covered in fabric 438 which surrounds flexible leaflets of the valve. In the disclosed embodiment, three access ports 434 are evenly distributed around the outer frame, passing through the cover fabric 438 between struts of the structural frame 436. Although not shown, aligned access ports will be provided in a lower end of the valve 430 so that a sensor lead 432 can be passed straight through from proximal to distal, or from the atrial to the ventricular sides of the valve.

Of course, more or less than three sets of aligned access ports 434 may be provided. The small nature of the access ports 434 reduces any regurgitation which might result, and fabric flaps may also be added to cover the access ports 434 upon back pressure of blood. Likewise, the access ports 434 might be configured to be normally closed, with the capacity for dilation, actuation or even cutting through marked areas to open them up. Additionally, the access ports 434 may have fluoro or echo marker bands around their perimeters to aid in passing the sensor lead 432 though them.

While the foregoing is a complete description of the preferred embodiments of the invention, various alternatives, modifications, and equivalents may be used. Moreover, it will be obvious that certain other modifications may be practiced within the scope of the appended claims.

What is claimed is:

1. A prosthetic heart valve delivery system, comprising:
a flexible access sheath having a lumen;
a proximal handle;
a delivery catheter extending distally from the proximal handle, the delivery catheter having an outer diameter sized to fit through the lumen of the access sheath, the delivery catheter also formed with a lumen extending therethrough;
an expandable prosthetic heart valve adapted to be crimped and positioned within the lumen of the delivery catheter along a distal end portion of the delivery catheter;
a tapered nose cone coupled to and projecting distally from a distal end of the delivery catheter in an extended state, the nose cone adapted to facilitate passage of the delivery catheter through the patient's vasculature, wherein the nose cone is formed of a series of nested layers which form a tapered elongated shape in the extended state and which may be collapsed longitudinally to a collapsed state for reducing contact with a wall of the heart; and
an inner catheter extending from the proximal handle through the lumen of the delivery catheter, through the prosthetic heart valve, and attached to the nose cone.

2. The system of claim 1, wherein the inner catheter is attached to a distal end portion of the nose cone and wherein the nose cone transitions to the collapsed state when the inner catheter is pulled.

3. The system of claim 1, wherein the nose cone is surrounded by a flexible cover.

4. The system of claim 1, wherein the nested layers are biased into the tapered elongated shape.

5. The system of claim 1, wherein the nose cone may be advanced beyond a distal end of the delivery catheter.

6. The system of claim 1, wherein the nested layers are nested in an angular manner that may collapse by rotating or retracting a pull wire.

7. The system of claim 6, wherein the nose cone is surrounded by a flexible cover.

8. The system of claim 1, wherein the flexible access sheath has a wall construction that enables conversion between a stiff configuration to a flexible configuration.

9. The system of claim 1, wherein the flexible access sheath has a wall construction that enables conversion between an extended configuration to an axially collapsed configuration.

10. A prosthetic heart valve delivery system, comprising:
a flexible access sheath having a lumen;
a proximal handle;
a delivery catheter extending distally from the proximal handle, the delivery catheter having an outer diameter sized to fit through the lumen of the access sheath, the delivery catheter also formed with a lumen extending therethrough;
an expandable prosthetic heart valve adapted to be crimped and positioned within the lumen of the delivery catheter along a distal end portion of the delivery catheter;
a tapered nose cone coupled to and projecting distally from a distal end of the delivery catheter in an extended state, the nose cone adapted to facilitate passage of the delivery catheter through the patient's vasculature, the nose cone being collapsible to a collapsed state for reducing contact with a wall of the heart, and wherein the inner catheter extends through the nose cone to a distal end thereof, and the nose cone is configured to invert upon itself when the inner catheter is pulled; and
an inner catheter extending from the proximal handle through the lumen of the delivery catheter, through the prosthetic heart valve, and attached to the nose cone.

11. The system of claim 10, wherein the nose cone is formed of an elastomeric material which may be inverted upon itself to the collapsed state.

12. The system of claim 10, wherein the nose cone is flat in the collapsed state.

13. The system of claim 10, wherein the nose cone is rounded in the collapsed state.

14. The system of claim 10, wherein the flexible access sheath has a wall construction that enables conversion between a stiff configuration to a flexible configuration.

15. The system of claim 10, wherein the flexible access sheath has a wall construction that enables conversion between an extended configuration to an axially collapsed configuration.

16. A prosthetic heart valve delivery system, comprising:
a proximal handle;
an elongate delivery catheter extending distally from the proximal handle, the delivery catheter sized for advancement through a lumen of an access sheath;
an expandable prosthetic heart valve adapted to be crimped and positioned along a distal end portion of the delivery catheter;
an inner catheter extending through an inner lumen of the delivery catheter; and
a nose cone coupled to a distal end portion of the inner catheter;
wherein the nose cone facilitates passage of the delivery catheter through the patient's vasculature;
wherein the nose cone is provided with a series of layers that form a tapered elongated shape during advancement through the patient's vasculature; and
wherein at least some of the layers may be repositioned by manipulation of the inner catheter for reducing a length of the nosecone, thereby reducing undesirable contact with a wall of the heart.

17. The system of claim 16, wherein the inner catheter is attached to a distal end portion of the nose cone and wherein the nose cone transitions to the collapsed state when the inner catheter is pulled.

18. The system of claim 17, wherein the nose cone is surrounded by a flexible cover.

19. The system of claim 18, wherein the nose cone is formed of a series of stacked nested layers which form a tapered elongated shape in the extended state, and which may be collapsed longitudinally to a collapsed state for reducing contact with a wall of the heart.

20. The system of claim 18, wherein the nose cone is formed of an elastomeric material which may be inverted upon itself to the collapsed state.
